(19) United States
(12) Patent Application Publication
(10) Pub. No.: US 2012/0301720 A1
(43) Pub. Date: Nov. 29, 2012

(54) METAL ISLAND COATINGS AND METHOD FOR SYNTHESIS

(75) Inventors: Wieland Koban, Mannheim (DE); Wolfgang Peukert, Markt Schwaben (DE); Robin Klupp Taylor, Forchheim (DE); Monica Distaso, Uttenreuth (DE); Huixin Bao, Erlangen (DE); Serhiy Vasylyev, Erlangen (DE)

(73) Assignee: BASF SE, Ludwigshafen (DE)

(21) Appl. No.: 13/510,073

(22) PCT Filed: Nov. 4, 2010
(86) PCT No.: PCT/EP2010/066788

§ 371 Date: May 16, 2012

(30) Foreign Application Priority Data
Nov. 16, 2009 (EP) 09176109.8

(51) Int. Cl. B22F 9/24; B32B 15/02; A61K 9/00; G01N 21/65; C09K 5/00; C09K 3/00; C08K 3/08; C09D 11/00; C22B 5/00; B01J 35/02

(52) U.S. Cl. 428/402; 427/256; 75/392; 424/400; 502/300; 252/71; 252/600; 252/1; 106/403; 106/31.13; 252/408.1

(57) ABSTRACT

The present invention relates to methods for synthesis of metallic island coatings with tunable island coverage and morphology on a variety of substrates. Particularly, the present invention relates to substrates coated with one or more metal islands and the use of said island-coated substrates.

The present invention relates to methods for synthesis of metallic island coatings with tunable island coverage and morphology on a variety of substrates. Particularly, the present invention relates to substrates coated with one or more metal islands and the use of said island-coated substrates.

Small particles with micrometer or nanometer dimensions find a wide range of applications in fields such as pigments, cosmetics, printing systems, optoelectronic materials and devices, biomedical diagnosis and clinical therapy systems and catalysis. Due to the manyfold application demands including physical function, chemical and thermal stability, environmental safety etc., particles with a composite morphology have been realised. Examples of such structures include core-shell particles—one material component coated by another—and composite particles—a plurality of smaller particles of one or more material components within a dense or porous matrix. In particular, the formation of coatings on particles represents a simple route to particle multifunctionality and stability. Many systems are known whereby particles can be coated to improve or extend their properties, e.g. by sol-gel techniques, adsorption of polymers or functional molecules, or gas-phase processes such as atomic layer deposition, aerosol pyrolysis or physical vapour deposition.

In certain cases it would be particularly desirable for a particle or particle coating to be asymmetrical in terms of its topology (distribution of substance). To this end, one can envisage particles of two or more substances adjoined or a core particle being partially or fully coated by one or more different substances or the coating being of inhomogeneous thickness. In the case of a particle wherein the surface properties substantially differ on either side of a particle, in the art this type of particle is sometimes referred to as a “Janus Particle” after the two-faced Roman God of Change of the same name. However, a more general case can be imagined whereby a particle is covered by an arbitrary number of islands of the same or differing size of a certain substance. In the context of the present invention, the term “asymmetrical particle” refers to this, more general case.

A reason for forming an asymmetrical particle or particle coating might be to provide asymmetrical surface functionality in order to assist the subsequent application of the particles, or their deposition, assembly or arrangement into films or self-standing structures. To this end, particles consisting of bimetallic coatings have been shown to demonstrate electrochemical propulsion due to differing local redox chemistry. It has also been reported that magnetic Janus particles can be trapped by laser tweezers and be provided with, in addition to the usual 3-dimension translational control, two orthogonal degrees of rotational movement. Furthermore, Janus particles consisting of a cap of magnetic material on a spherical core can be used to self-assemble into linear structures such as zig-zag chains which are bistable, they redisperse into single particles, These might have applications in fluids with tunable rheological properties. The reversible aggregation of small nanoparticles using pH sensitive inorganic/organic Janus particles was also demonstrated. It is also known that Janus particles, due to their multifold surface functionality can replace conventional surfactants in the stabilization of emulsions.

A further application of Janus particles might be to provide limited access of a chemical species diffusing to or away from or adsorbing to or desorbing from the core or even removal of the core to provide access to or from a hollow cavity. To this end, applications in biological polarized targeting are being envisaged and hollow biocompatible capsules with a single entrance hole have been reported.

A yet further application of asymmetrically coated particles might be to provide a novel electromagnetic or other physical function associated with the coating's asymmetry. Examples fitting to this class include IR extinguishing pigments based on metal—Cu, Ag, Au, Al—nanocaps, light-bending plasmonic nanocups—metamaterial superlens—and ultrasensitive biomolecular detection through the surface-enhanced Raman scattering effect at metal semishells. Janus particles are also promising for bistable display devices—electronic paper—whereby particles with differently coloured sides are rotated by an electric or magnetic field.

It will be appreciated that a significant class of asymmetrical particles comprises asymmetrical coatings on core particles. It will be appreciated by someone knowledgeable in the art that for maximum commercial benefit of asymmetrical particle coatings, techniques to produce particles with controlled coverage must be developed. In other words, the coating process should allow the coating to cover a proportion of the core particle's surface ranging from a very low to complete coverage. A further, important requirement is that techniques to produce asymmetrical particle coatings are scalable.

A drawback of most particle coating techniques taking place in homogeneous media, whether operating in the liquid or gas phase is that they act indiscriminately on the core particle i.e. they form a conformal shell around the particle. This means that neither the coating's coverage nor its thickness is tunable locally.

Coating methods are known which can provide single island coatings with limited possibility for tuning the coating coverage and thickness inhomogeneity.

US 2002/0160195 A1 discloses partial coverage metal nanoshells and a method for making the same. This method makes use of a substrate which chemically binds core particles through functional linker molecules. The remaining exposed portion of the core can be functionalized and coated with a metal using known techniques. Limited tunability of the coating coverage is possible by modifying the functional linker molecules. The region of partial coverage is continuous i.e. only one metal island per core particle can be formed according to this invention.

US 2003/0215638 A1 discloses a method for producing reduced symmetry nanoparticles by masking 10-90% of the surface area of the core and applying a conducting shell to the exposed part. The region of coating coverage is continuous i.e. only one conducting island can be formed per core particle according to this invention.

US 2008/0234394 A1 deals with a method for forming Janus particles consisting of a molecular coating on the core particle with coverage between 10 and 50%. This method also makes use of a phase boundary, in this case the surface of an emulsion droplet. Particles immobilized at the droplet surface by droplet solidification are effectively masked on their inward-facing side. This allows functionalization of the exposed surface. The region of coating coverage is continuous i.e. only one molecular coating island can be formed per core particle according to this invention.

US 2006/0159921 A1 is drawn towards an inhomogeneous nanoparticle coating on a polyelectrolyte aggregate. The aggregate, formed by agglomerating polyelectrolyte molecules in the presence of a counterion leads to attraction of charged nanoparticles to certain sites on the aggregate surface.

Coating methods are also known which provide substantially continuous metallic coatings on non-metallic cores. The most relevant example is U.S. Pat. No. 6,344,272 B1 which claims a non-metallic core coated with a metallic shell which covers between 10% and 100% of the area of the non-metallic core. The coverage of the core and thickness of the shell affects strongly the absorption and scattering of radiation incident on the particle. However, it is to be understood from the preferred embodiments disclosed in U.S. Pat. No. 6,344,272 B1 that in the case of coverage less than 100%, metallic islands produced by the method are homogeneously distributed and have arbitrary (non-controlled) topology. Furthermore, the techniques to produce these metallic coatings are not easily scaled-up due to the need for particle functionalization with linker molecules and nanoparticles.

Asymmetrical particles and coatings are also known from the literature. Most work can be attributed to one of three categories:
Use of a phase boundary to mask or shadow part of the core particle
Use of functional coating molecules which competitively phase-separate to form a patchy or completely two-faced particle
Use of combinations of crystalline materials where the coating material preferentially nucleates on one part of the core particle

None of the works disclosed in the above patents and publications provide substrates which are coated with more than one metallic island. Furthermore, where one metallic island is provided, the tunability of the island's size and morphology is limited and the techniques used are not easily scaled-up.

Although techniques are known to produce continuous and discontinuous metallic films on planar non-metallic substrates, non-templated, non-lithographic techniques to produce isolated metallic islands on such substrates with controlled island size and morphology are unknown.

Therefore it is an object of the invention to provide methods for synthesis of metallic island coatings with tunable island coverage and morphology on a variety of substrates. It is a further object of the invention to provide methods for synthesis of metallic island coatings on non-metallic substrates avoiding the need for a phase boundary, mask, phase-separating molecule or suitable material pairing. It is a further object of the invention to provide substrates coated with one or more metal islands.

The problem underlying the invention is solved by a method for synthesis of non-metallic substrates coated with one or more metallic islands, comprising
(a) Providing a substrate,
(b) Treating the substrate with a polar solvent for at least 10 minutes, wherein the polar solvent comprises one or more compounds selected from the group consisting of metal ions, metal ions and complexing agents and metal complexes, and
(c) Treating the substrate subsequent to step (b) with one or more reducing agents.

Accordingly, the present invention provides a first method for forming a metallic island coating on a substrate where said substrate has been functionalized via homogenous chemical techniques (i.e. absence of deliberate patterning) and is treated in solution.

According to this first preferred embodiment of the invention, a substrate that is preferably a non-metallic substrate with an average radius of curvature between 5 nm and infinity is provided in step (a) and treated in a polar solvent which comprises one or more compounds selected from the group consisting of metal ions, metal ions and complexing agents and metal complexes. Following treatment for at least 10 minutes, preferably for at least 20 minutes, more preferably for at least 30 minutes, most preferably for at least 60 minutes, reducing agents, and optionally, additives are added in step (c). Depending on the type of metal, the total surface area of the non-metallic substrate, concentration of metal ions or complexes, ageing time in presence of said metal ions or complexes, type of reducing agent or agents, nature of additives, order of addition of reducing agent or agents and additives, metallic island coatings are formed with varying surface coverage and coating thickness.

According to a further aspect of the invention, a second embodiment is provided for synthesis of non-metallic substrates coated with one or more metallic islands, comprising
(a) Providing a substrate,
(a′) Treating the substrate with a polar solvent that optionally comprises at least one metal complexing agent for at least one minute,
(b) Treating the substrate with a polar solvent for at least 1 minute, wherein the polar solvent comprises one or more compounds selected from the group consisting of metal ions, metal ions and complexing agents, metal complexes, and metal nanoparticles, and
(c) Treating the substrate subsequent to step (b) with one or more reducing agents.

According to this method of the invention, a substrate, that is preferably a non-metallic substrate with an average radius of curvature between 5 nm and infinity is provided in step (a) and treated in a polar solvent to which a metal complexing agent is optionally added in step (a′). Following treatment for at least 1 minute, preferably for at least 10 minutes, more preferably for at least 30 minutes, most preferably for at least 60 minutes, the non-metallic substrate is treated in a polar solvent comprising one or more compounds selected from the group consisting of metal ions, metal ions and complexing agents, metal complexes, and metal nanoparticles in step (b). Following treatment for at least 1 minute, preferably for at least 10 minutes, more preferably for at least 30 minutes, most preferably for at least 60 minutes, reducing agents, and optionally, additives are added in step (c). Depending on the type of metal, the total surface area of the non-metallic substrate, concentration of metal ions or complexes, ageing time in presence of said metal ions or complexes, type of reducing agent or agents, nature of additives, order of addition of reducing agent or agents and additives, metallic island coatings are formed with varying surface coverage and coating thickness.

In a further embodiment of the invention, a substrate that is preferably a non-metallic substrate with an average radius of curvature between 5 nm and infinity is provided in step (a) and is treated with a first solvent in step (a). This treatment may be carried out in a polar or non-polar solvent. This treatment may be carried out at any temperature or pressure. The duration of this treatment should be at least 1 minute, preferably at least 10 minutes, more preferably at least 30 minutes, most preferably at least 60 minutes. The non-metallic substrate is then treated in a polar solvent comprising one or more compounds selected from the group consisting of metal ions, metal ions and complexing agents, metal complexes, and metal nanoparticles in step (b). This treatment may be carried out at any temperature or pressure. The duration of this treatment should be at least 1 minute, preferably for at least 10 minutes, more preferably for at least 30 minutes, most preferably for at least 60 minutes. Following this, the non-metallic substrate is treated in step (c), preferably in a second polar solvent comprising one or more compounds selected from the group consisting of metal ions or metal complexes, reducing agents and additives. Depending on the type of metal, the total surface area of the non-metallic substrate, concentration of metal ions or complexes, ageing time in presence of said metal ions or complexes, type of reducing agent or agents, nature of additives, order of addition of reducing agent or agents and additives, metallic island coatings are formed with varying surface coverage and coating thickness.

In yet another embodiment of the invention, a substrate that is preferably a non-metallic substrate with an average radius of curvature between 5 nm and infinity is provided in step (a) and is treated in step (b) in a first polar solvent comprising metal ions or metal ions and complexing agents or metal complexes or metal nanoparticles. This treatment may be carried out at any temperature or pressure. The duration of this treatment should be between 1 minute and 48 hours, preferably between 10 minutes and 40 hours, more preferably between 20 minutes and 24 hours. Following this, the non-metallic substrate is treated in step (c), preferably in a second polar solvent comprising comprising one or more compounds selected from the group consisting of metal ions, metal complexes, reducing agents and additives. Depending on the type of metal, the total surface area of the non-metallic substrate, concentration of metal ions or complexes, ageing time in presence of said metal ions or complexes, type of reducing agent or agents, nature of additives, order of addition of reducing agent or agents and additives, metallic island coatings are formed with varying surface coverage and coating thickness.

An even still further preferred embodiment of the invention step (b) in any of the abovementioned preferred embodiments, but in particular in the first preferred embodiment, comprises treating the substrate with a polar solvent comprising one or more compounds selected from the group consisting of metal ions, metal ions and complexing agents and metal complexes, at a temperature from 35 to 95° C., preferably from 40° C. to 90° C., more preferably from 45° C. to 80° C. and in particular 50 to 70° C. The duration of this treatment should be between 1 minute and 120 minutes, preferably between 10 and 80 minutes and most preferably between 30 and 60 minutes. Following this, at the same temperature as in step (b) the non-metallic substrate is treated in step (c), in a polar solvent comprising one or more reducing agents and one or more compounds selected from the group consisting of metal ions, metal ions and complexing agents, metal complexes and additives.

According to a yet further preferred embodiment of the invention the substrate treated in step (b) is coated with one or more molecules and/or macromolecules at any step prior step (c). According to this embodiment a substrate that is preferably a non-metallic substrate with an average radius of curvature between 5 nm and infinity is provided in step (a) and coated with at least one molecule or macromolecule which contains units capable of interacting with metal ions so as to form at least one metal nanoparticle with a diameter smaller than at most 100 nm, said metal being immobilized on the surface of the non-metallic particle.

According to this embodiment of the invention, the coating with one or more molecules and/or macromolecules may be carried out before, simultaneously or after treatment of the non-metallic substrate in step (a′) or step (b) at any temperature or pressure for at least 1 minute, preferably for at least 10 minutes. Furthermore, in this embodiment step (b) may be optionally omitted. In optional step (b) the non-metallic substrate is treated in a polar solvent comprising metal ions or metal ions and complexing agents or metal complexes or metal nanoparticles. This treatment may be carried out at any temperature or pressure. The duration of this treatment should be at least 1 minute, preferably at least 10 minutes. Following this, the non-metallic substrate is treated in step (c) in a polar solvent to which metal ions or metal complexes, reducing agents, additives are added. Depending on the type of metal, the total surface area of the non-metallic substrate, concentration of metal ions or complexes, ageing time in presence of said metal ions or complexes, type of reducing agent or agents, nature of additives, order of addition of reducing agent or agents and additives, metallic island coatings are formed with varying surface coverage and coating thickness.

According to a still further preferred embodiment of the invention the substrate treated in step (b) is coated with nanoparticles with an average particle size smaller than 100 nm, at any step prior step (c).

According to this embodiment of the invention, a substrate that is preferably a non-metallic substrate with an average radius of curvature between 5 nm and infinity is provided in step (a) and coated with at least one metal nanoparticle with a diameter smaller than 100 nm, more preferably smaller than 50 nm, even more preferably smaller than 10 nm, most preferably smaller than 5 nm. The coating with at least one metal nanoparticle may be carried out before, simultaneously or after treatment of the non-metallic substrate in step (a′) or step (b) at any temperature or pressure for at least 1 minute, preferably more than 10 minutes. Furthermore, in this embodiment step (b) may be optionally omitted. In optional step (b) the non-metallic substrate is treated in a polar solvent comprising metal ions or metal ions and complexing agents or metal complexes or metal nanoparticles. This treatment may be carried out at any temperature or pressure. The duration of this treatment should be at least 1 minute, preferably at least 10 minutes. Following this, the non-metallic substrate is treated in step (c) in a polar solvent to which metal ions or metal complexes, reducing agents, additives are added. Depending on the type of metal, the total surface area of the non-metallic substrate, concentration of metal ions or complexes, ageing time in presence of said metal ions or complexes, type of reducing agent or agents, nature of additives, order of addition of reducing agent or agents and additives, metallic island coatings are formed with varying surface coverage and coating thickness.

According to an even still further preferred embodiment of the invention step (a) in any of the abovementioned preferred embodiments comprises providing a substrate that is preferably a non-metallic substrate and treating said substrate with a non-polar solvent.

An even still further preferred embodiment of the invention step (a) in any of the abovementioned preferred embodiments, but in particular in the first preferred embodiment, comprises providing a substrate that is preferably a non-metallic substrate which is at first treated by calcination at 500-1100° C., more preferably 600-1000° C., most preferably at 800-1000° C.

The combination of the first embodiment of the invention with the embodiment concerning the calcination of the substrate in step (a) and the embodiment concerning the treatment with a polar solvent at a temperature from 35 to 95° C. in steps (b) and (C) is in particular preferred.

A particular preferred embodiment comprises a method for synthesis of non-metallic substrates coated with one or more metallic islands, comprising
(a) Providing a substrate, which is at first treated by calcination at 500-1100° C., more preferably 600-1000° C., most preferably at 800-1000° C.,
(b) Treating the substrate with a polar solvent for at least 1 minute, wherein the polar solvent comprises one or more compounds selected from the group consisting of metal ions, metal ions and complexing agents and metal complexes, at a temperature from 35 to 95° C., preferably from 40° C. to 90° C., more preferably from 45° C. to 80° C. and in particular from 50 to 70° C. and
(c) at the same temperature treating the substrate subsequent to step (b) with a further polar solvent, wherein the further polar solvent comprises one or more reducing agents and one or more compounds selected from the group consisting of metal ions, metal ions and complexing agents, metal complexes and additives.

It should also be appreciated that all possible orders of addition and times between addition of metal ions or complexes, reducing agents and additives in step (c) solvent comprise further embodiments of the invention.

In another even still further preferred embodiment of the invention, in any of the abovementioned preferred embodiments, the substrate is subjected a washing step prior to step (a′) and/or (b) and/or (c). It will be appreciated by someone skilled in the art that between treatments and coating steps, separation of the non-metallic substrate from the solvent and washing with the solvent and the next to be used polar solvent is beneficial. Where the polar solvents are different this separation and washing is necessary. When the non-metallic substrate is a particle, techniques of separation and washing include sedimentation, centrifugation, evaporation or filtration.

According to an even still further preferred embodiment of the invention, in any of the abovementioned preferred embodiments, subsequent to step (c) the substrate is removed by chemical or heat treatment. If the substrate is silica based, the substrate is removed by chemical treatment in particular by acid dissolution with aqueous hydrogen fluoride. If the substrate is a polymer, the substrate can be removed by chemical treatment with a suitable solvent or preferably the polymer substrate can be removed by heat treatment. Said solvents suitable for dissolving the polymer depend on the type of polymer and are known to someone skilled in the art. The temperature suitable for removal of the polymer by decomposing or evaporating the polymer substrate depends on the type of polymer and is known to someone skilled in the art. The metallic islands obtainable according to this embodiment retain their shape. Said metal islands can advantageously be used for various applications such as drug delivery systems, heat management, thermal management, in diagnostics, as a surface-enhanced Raman spectroscopy agent, as pigment, as catalyst, in a light detecting device, in an electronic ink or as chemical sensing device.

It will be appreciated by someone skilled in the art that certain metal ions and complexes, in the company of certain ligands undergo photochemical reactions. In some embodiments of the invention, some or all of the treatments described above may be carried out in the presence or absence of light. Light may include here, ambient sun or room (fluorescent or incandescent) light or light comprising spectral lines e.g. from a mercury or xenon lamp.

In the context of the invention the non-metallic substrate provided in step (a) may be selected from the group consisting of metal oxides (for example SiO₂, TiO₂, Al₂O₃, ZrO₂, In₂O₃, Fe₂O₃, Fe₃O₄), silicates (e.g. mica), ferrites, metal sulphides, metal nitrides, metal carbonates, metal hydroxycarbonates, polypeptides, proteins, nucleic acids, glass (for example fused silica), ceramics (for example TiO₂, Al₂O₃, ZrO₂), carbon (for example carbon nanoparticles, single- and multiwalled-nanotubes) and polymers (for example, polystyrene, polypropylene, latex, polyacrylamide).

It is to be understood that it is the surface region (to a depth of at least 1 nm) of the substrate that must be non-metallic. It will be therefore be appreciated that the substrate could comprise a metallic underlayer coated with a layer or multilayer of material whereby the outer layer is non-metallic.

The substrate may have one of a variety of shapes, such as spherical, ellipsoid, rod-like, fibrous helical, and oblate, among others. The substrate may be solid or hollow.

The non-metallic substrate may optionally be provided with a coating with polymeric, oligomeric or molecular material (e.g. for the purpose of stabilization or for further functionalization). The non-metallic substrate may be substantially porous or substantially non-porous. Without loss of generality, the non-metallic substrate could comprise a nanoparticle with a diameter in the range of 10 to 500 nm, a microparticle with a diameter in the range of 0.5 to 100 micrometers, or a macroparticle with a diameter greater than 100 micrometers or a substantially planar surface.

According to a preferred embodiment, the non-metallic substrate comprises SiO₂ particles with a mean diameter of less than 1000 nm, preferred with a diameter in the range of 10 to 500 nm. These particles are preferably synthesised by the hydrolysis and condensation of tetraethoxysilane by base catalysis (Stöber method) and separated and dried.

According to a further preferred embodiment, the non-metallic substrate comprises porous SiO₂ particles with a mean diameter of less than 1000 nm. These particles are preferably synthesised by the hydrolyisis and condensation of tetraethoxysilane by base catalysis in the presence of a pore-former such as cetyltrimethylammonium bromide (CTAB). In a yet further preferred embodiment, the non-metallic substrate comprises porous amorphous titania particles with a size less than 1000 nm, for example those provided by Corpuscular Inc.

In general, the polar solvent used in steps (a), (a′), (b) and (c) may be any polar solvent known from the art. According to a preferred embodiment of the invention, the polar solvent is selected from the group consisting of water, tetrahydrofuran, 1,4 dioxane, dimethylsulfoxide, dimethylformamide and C₁ to C₆ alcohols such as methanol, ethanol, n-propanol, 2-propanol, n-butanol, iso-butanol and tert-butanol, or a mixture of two, three, four or more of the aforementioned solvents. According to a more preferred embodiment, the polar solvent is water or 1,4 dioxane.

In general, the non-polar solvent used in step (a) may be any non-polar solvent known from the art.

The metal ion or the metal present in the metal complexes or in the metal nanoparticles in step(s) (b) and/or (c) is at least one metal selected from the group consisting of Ag, Au, Cu, Pt, Pd, Ru, Rh, Fe, Ti, Al, Ni, Co, Mg, Mn, Zn and Cr. In a preferred embodiment of the invention, the metal is selected from the group of Ag and Au. The proportion of the metal ion or metal complex in the solution(s) used in process step (b) and (c) may vary over wide ranges. In general, the proportion of the metal is in the range from 1×10⁻⁴ to 10% by weight, preferably in the range from 5×10⁻⁴ to 5% by weight and most preferably in the range from 5×10⁻³ to 1% by weight, based on the solution provided in process step (b) and (c).

Sources of metal ions or metal complexes for the treatment steps (b) and/or (c) include inorganic or organic salts.

Inorganic salts in the context of the present invention are, for example, chlorides, sulfates and nitrates, provided that these combinations of inorganic anions and the particular metal cations exist. Organic salts in the context of the present invention are salts of carboxylic acids, for example formates, acetates, citrates, alkoxides and acetylacetonates with the corresponding metals, provided that combinations of organic anions and a particular metal cation exist.

Preferred sources of metal ions or metal complexes include but are not limited to Silver nitrate, Ammoniacal silver nitrate Ag(NH₃)₂NO₃, silver-alkanalamine complexes, Silver carbonate, silver sulphate, silver tosylate, silver acetate, silver methanesulfonate, silver trifluoroacetate, silver pentafluoropropionate, chloroauric acid, gold(III) chloride, chloroplatinic acid, palladium acetate. It should be appreciated that the metal ions or metal complexes present in the treatment in the polar solvent in steps (b) and (c) may have the same type and concentration or may have different types and concentrations.

In the above embodiments, the metal complexing agent or complexing agent is generally an organic or inorganic compound which is capable of complexing metal cations.

According to preferred embodiments of the invention, the metal complexing agent is at least one selected from the group consisting of monoethanolamine, diethanolamine and triethanolamine. Other possible metal complexing agents include tertiary amines and molecules containing the following known metal-chelating groups: phenol, carbonyl, carboxylic, hydroxyl, ether, phosphoryl, amine, nitro, nitroso, azo, diazo, nitrile, amide, thiol, thioether, thiocarbamate and bisulphite. Complexing agents are generally selected from monocarboxylic acids, dicarboxylic acids, tricarboxylic acids, hydroxycarboxylic acids, ketocarboxylic acids, diketones, amino acids, aminopolycarboxylic acids, polymer-bound carboxylic acids, amines, diamines, ammonia, nitrate ions, nitrite ions, halide ions and hydroxide ions, or a salt of the aforementioned acids. The complexing agents are preferably monocarboxylic acids such as formic acid, acetic acid and propionic acid, dicarboxylic acids such as oxalic acid, malonic acid, succinic acid, glutaric acid and tartaric acid, tricarboxylic acids such as citric acid, hydroxycarboxylic acids such as tartaric acid, ketocarboxylic acids such as pyruvic acid, diketones such as acetylacetone, and aminopolycarboxylic acids such as ethylenediaminetetraacetic acid, or a salt of the aforementioned acids. In a further preferred embodiment, the complexing agent is a compound selected from ethylenediaminetetraacetic acid, triethanolamine, formic acid, acetic acid, pantothenic acid, folic acid, biotin, arachidonic acid, malonic acid, α-aminobutyric acids, β-aminobutyric acids, γ-aminobutyric acids, glutathione, isocitric acid, cis- and trans-aconitic acid, hydroxycitric acid, nicotinic acid, benzoic acid, oxalic acid, mesoxalic acid, oxalacetic acid, succinic acid, sorbic acid, propanetricarboxylic acid, crotonic acid, itaconic acid, acrylic acid, methacrylic acid, mesaconic acid, phenylacetic acid, salicylic acid, 4-hydroxybenzoic acid, cinnamic acid, mandelic acid, 2-furancarboxylic acid, acetoacetic acid, glucuronic acid, gluconic acid, glucaric acid, glyceric acid, glycolic acid, butyric acid, isobutyric acid, hexanoic acid, heptanoic acid, octanoic acid, decanoic acid, undecanoic acid, dodecanoic acid, tetradecanoic acid, palmitic acid, stearic acid, methacrylic acid, urocanic acid, pyrrolidonic acid, glutaric acid, adipic acid, pimelic acid, suberic acid, azelaic acid, sebacic acid, phthalic acid, terephthalic acid, 2- and 3-hydroxybenzoic acid, lactic acid and citric acid, or a salt of the aforementioned acids.

In one embodiment of the invention, the molar ratio of metal ion to complexing agent in the solution used in process step (b) or (c) is in the range from 1:0.1 to 1:500, preferably in the range from 1:0.5 to 1:100.

The proportion of the metal complexing agent in process step (a′) may vary over wide ranges. In general, the proportion is in the range from 0.1 to 20% by weight, preferably in the range from 0.5 to 10% by weight and more preferably in the range from 1 to 5%.

In process step (c) the pre-treated substrate is treated with at least one reducing agent. Typical reducing agents include, but are not limited to formaldehyde, hydrated electrons, sodium citrate, L-ascorbic acid, glucose, fructose, sodium borohydride, potassium borohydride, hydroquinone, catechol, Li(C₂H₅)H, glyoxal, formic acid, glyceraldehydes, glycolaldehyde dimer, hydroxylamine, hydrogen gas, glyoxal trimeric dehydrate, or mixtures thereof.

The proportion of the reducing agent in process step (c) may vary over wide ranges. In general, the proportion is in the range from 0.01 to 10% by weight, preferably in the range from 0.1 to 5% by weight and more preferably in the range from 0.5 to 1% by weight.

The solvents used in process steps (c) may further comprise one or more additives. Typical additives to support the formation of zero valent metal include acids and bases, organic polymers, oligomers and molecules. In a particular embodiment, where metal island coatings of silver or palladium are to be formed, these additives may include, but are not limited to bases such as sodium hydroxide, ammonium hydroxide, methylamine, dimethylamine, trimethylamine, ethylamine, triethylamine, ethanolamine, diethanolamine, triethanolamine, isopropylamine, ethylenediamine, dimethylethylendiamine, tetramethylethylendiamine. In a particular embodiment, where metal island coatings of gold are to be formed, these additives may include, but are not limited to potassium carbonate.

The proportion of the additives in process step (c) may vary over wide ranges. In general, the proportion is in the range from 0.0001 to 20% by weight, preferably in the range from 0.001 to 10% by weight and more preferably in the range from 0.01 to 5% by weight.

One of the above embodiments requires the non-metallic substrate provided in process step (a) to be coated with molecules or macromolecules which preferably contain units capable of interacting with metal ions so as to form complexes or at least one metal nanoparticle with a diameter smaller than at most 100 nm, said metal being immobilized on the surface of the non-metallic substrate.

Such molecules or macromolecules may contain functional units which include, but are not limited to anhydride, carboxylic acid, dicarboxylic acid, ethylene glycol groups. Suitable molecules and macromolecules include, but are not limited to polyacids and polyelectrolytes such as Poly(styrene sulfonic acid), Poly(2-acrylamido-2-methyl-1-propane sulfonic acid), Polyvinyl phosphonic acid), Poly(sodium, 4-styrene sulfonate), Poly(methacrylic acid), Poly(acrylic acid), Poly(diallyldimethyl ammonium chloride); copolymers such as Poly(styrene-co-maleic anhydride), Poly(styrene-co-maleic acid), Poly(maleic anhydride), poly(maleic acid), poly(ethylene-maleic anhydride), poly(ethylene maleic acid), Poly(N-vinyl-2-pyrrolidone-co-vinyl acetate), Poly(N-vinyl-2-pyrrolidone), Poly(2-ethyl-2-oxazoline), Poly(ethylene oxide) , poly(2,6-dimethyl-1,4-phenylene oxide), Poly(vinyl alcohol), poly(ethylene oxide), poly(ethylene oxide)-poly(propylene oxide)-poly(ethylene oxide) block co-polymer (Pluronic), poly(propylene oxide)-poly(ethylene oxide)-poly(propylene oxide) block co-polymer, poly(ethylene oxide-styrene oxide) block co-polymer, PDMS-graft-PEO, amphiphilic multiblock copolymers consisting of alternating polyethylene oxide and aliphatic units. Included are also polymers, oligomers and molecules capable of forming a crown-ether structure.

In the above embodiments, treatment or coating with metal nanoparticles is sometimes prescribed. Such nanoparticles have a diameter less than at most 100 nm. The metal nanoparticle may comprise the same metal as the metal ion or complex treated in the polar solvents. The nanoparticle may be bare or coated with stabilizing/functionalizing small molecules, oligomers or polymers. Typical materials for the nanoparticle include, but are not limited to Au, Ag, Cu, Pt, Pd, Ru, Fe, Ti, Zn, Al, Ni, Co, Mg, C, Si, Ge, In₂O₃, In₂O₃:Sn, Sn₂O₃ and Sn₂O₃.

The present invention further refers to a non-metallic substrate coated with one or more metallic islands obtainable by a process according to any of the methods described beforehand.

The present invention further refers to a non-metallic substrate coated with one or more metallic islands obtainable by a process according to any of the methods described beforehand and subsequently coated, by any method, with a top layer or any material.

The present invention further refers to a non-metallic substrate coated with one or more metallic islands comprising a non-metallic particle wherein the particle is coated with one or more metal islands and wherein the particle has an arbitrary shape and wherein the largest dimension of the particle is smaller than 50 μm.

The present invention further refers to a non-metallic substrate coated with one or more metallic islands comprising a non-metallic particle wherein the particle is coated with one or more metal islands and a top layer of any material wherein the particle has an arbitrary shape and wherein the largest dimension of the particle is smaller than 50 μm.

The morphology of the metal islands produced according to the above embodiments may comprise features of one or more of the island characteristics shown schematically in FIGS. 49 to 52. Furthermore, the sizes and shapes of the metal islands may be predominantly similar (monodispersed islands) or may be significantly dispersed (polydispersed islands). Without loss of generality, islands produced according to the above embodiments are expected to possess a longest dimension of at least 5 nm and at most 10 micrometres and are expected to have a thickness at the thinnest point of at least 1 nm and at the thickest point of at most 5 micrometres, provided the substrate dimensions permit this. The surface coverage of the islands expected to be achieved according to the above embodiments can be such that in the case of the non-metallic substrate being a particle, it ranges from a single island on the particle right up to a complete coverage (islands overlapping). In the case of the substrate having a high radius of curvature or for substantially flat substrates, the average separation of the islands achieved according to the above embodiments will range from 100 micrometres to 0 micrometres (islands overlapping).

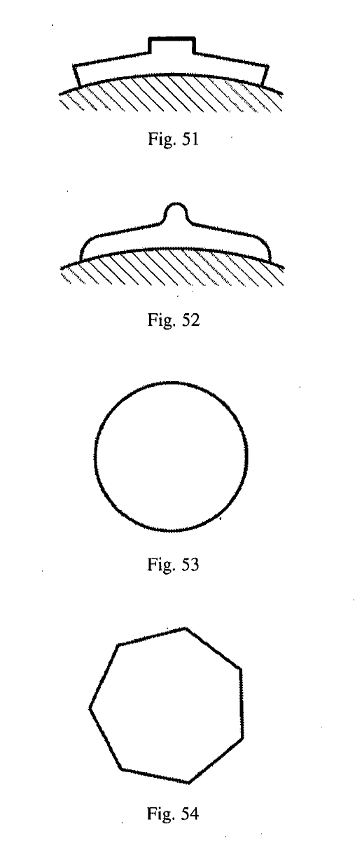

It is a further aspect of the present invention to provide a non-metallic substrate with a radius of curvature between 5 nm and infinity. This non-metallic substrate is covered by at least one metal island, said island having a thickness at its edges of at most the same thickness at the centre of the island (FIG. 49). In one embodiment of the invention the thickness may vary linearly from the centre of the island to the edge (FIG. 50). In another embodiment of the invention the thickness may vary non-linearly from the centre of the island to the edge. In a further embodiment of the invention, the thickness may vary as a step function (decreasing) from the centre of the island (FIG. 51). In a yet further embodiment, the thickness may be substantially uniform with a semi-circular asperity at the centre of the island, said semi-circular asperity having a radius of curvature of at least half the thickness of film at the edge of the island (FIG. 52).

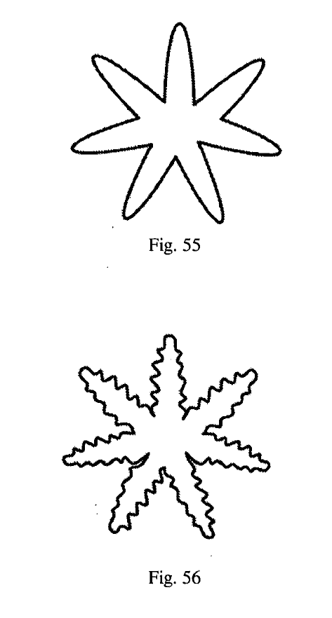

In plan view the island may appear circular (FIG. 53), ellipsoidal, prismatic (FIG. 54) or dendritic (FIGS. 55 and 56). The circular-equivalent diameter of the islands is at most 1 micrometer. Also claimed is at least one metallic island on a substrate surrounded by at least one satellite island (FIG. 57), radially separated from the mother island. Said satellite islands may have any thickness, preferably a similar thickness to the edge of the mother island.

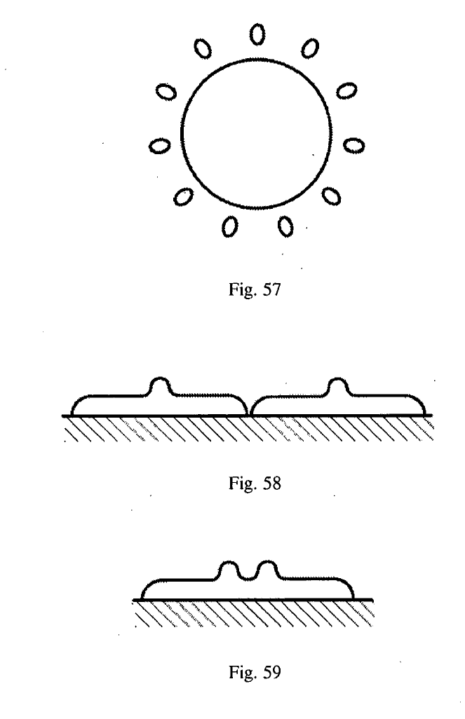

When more than one island is present on the non-metallic substrate, the islands may possess substantially similar morphologies and similar dimensions or may have either different morphologies or different dimensions or both. The islands may be touching at their outer edges (FIG. 58) or may be physically separate. They may also be merged so that their central regions are touching, overlapping or have a separation less than the sum of the equivalent diameters of two islands (FIG. 59).

Islands may be distributed on a substrate so that planes of symmetry exists or may be distributed so that no planes of symmetry exist.

It is a further aspect of the invention to provide a substrate capable of releasing metal ions. In such embodiments it is not necessary to carry out all of the steps as detailed above. In general, the process may cease following the step (b). In one embodiment, the metal provided is silver and the release of the silver ions is beneficial to, for example, antimicrobial applications.

It is a further aspect of the invention to provide a material which significantly extinguishes near-infrared radiation. In such embodiments, the coating metal is gold, silver or copper.

With the aid of the present invention, various possibilities arise for the production of the non-metallic substrates coated with one or more metallic islands. The resulting particles have a multitude of interesting properties. They are therefore promising new materials for various applications in as drug delivery systems, heat management, thermal management, in diagnostics, as pigment, as catalyst, in a light detecting device, in an electronic ink or as chemical sensing device.

The present invention is illustrated in detail by the examples cited and discussed below:

### EXAMPLE 1

### Asymmetrical Silver Coatings on Silica Particles Treated in Different Solvents

Treatment of Silicon Dioxide Particles in Polar Solvents

Silica particles (Monospher 500, Merck) were used both, as supplied and also after calcination at 800° C. for 24 hours.

The following silica suspensions were prepared:

| Code | Particle | Mass | Solvent | Volume of solvent |
|---|---|---|---|---|
| A1 | Silica (untreated) | 0.5 g | Pure water | 10 mL |
| A2 | Silica (calcined) | 0.5 g | Pure water | 10 mL |
| B1 | Silica (untreated) | 0.5 g | 1,4-Dioxane | 10 mL |
| B2 | Silica (calcined) | 0.5 g | 1,4-Dioxane | 10 mL |
| C1 | Silica (untreated) | 0.5 g | Tetrahydrofuran | 10 mL |
| C2 | Silica (calcined) | 0.5 g | Tetrahydrofuran | 10 mL |

Treatment of Silica Particles with Silver Complex.

1 mL aliquots of silica suspensions that had been treated for 10 days in polar solvents as described above were removed and were washed 3 times by centrifugation and high purity water redispersal. The final volume in each case was 1 mL. An ethanolamine-silver complex was obtained by adding 250 μL ethanolamine dropwise to a 250 μL aliquot of 2.8M silver nitrate aqueous solution. A 60 μL aliquot of this ethanolamine-silver complex solution was added to 1 mL of the silica suspension. After stirring for 1 hour, the suspensions were washed 3 times by centrifugation and high purity water redispersal. The final volume in each case was 1 mL.

Formation of Asymmetrical Silver Coatings on Silica Particles

Prior to treatment, a small amount of the silver-ethanolamine complex treated silica suspension (hereafter referred to as the “seed”) was diluted by a factor of 10. Coating experiments comprised adding aliquots (100 μL, 50 μL or 20 μL) of this diluted seed to 5 mL of a 100 μM silver nitrate solution under vigorous stirring followed by addition of 100 uL 37% aqueous formaldehyde solution and 100 μL 8% aqueous ammonia solution.

Small aliquots of freshly produced asymmetrical silver-silica suspensions were dried on silicon wafers. These were investigated by SEM. Furthermore, the optical extinction spectra of freshly produced silver-silica suspensions were obtained.

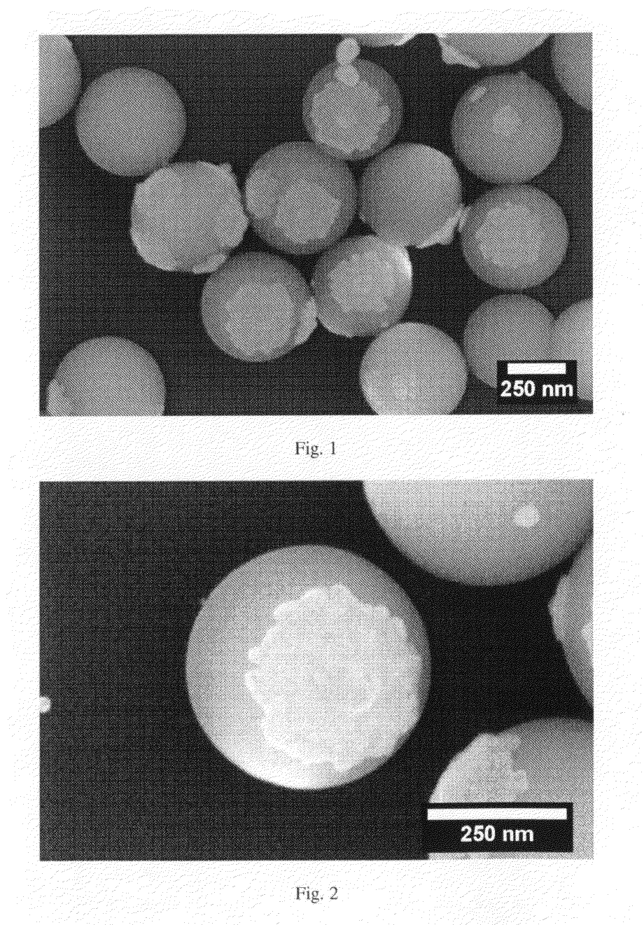

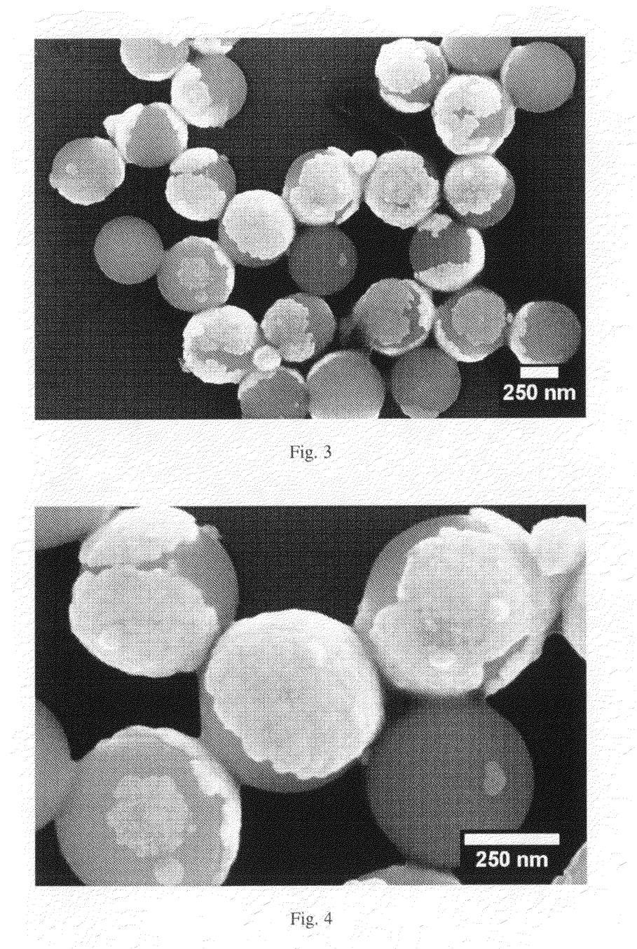

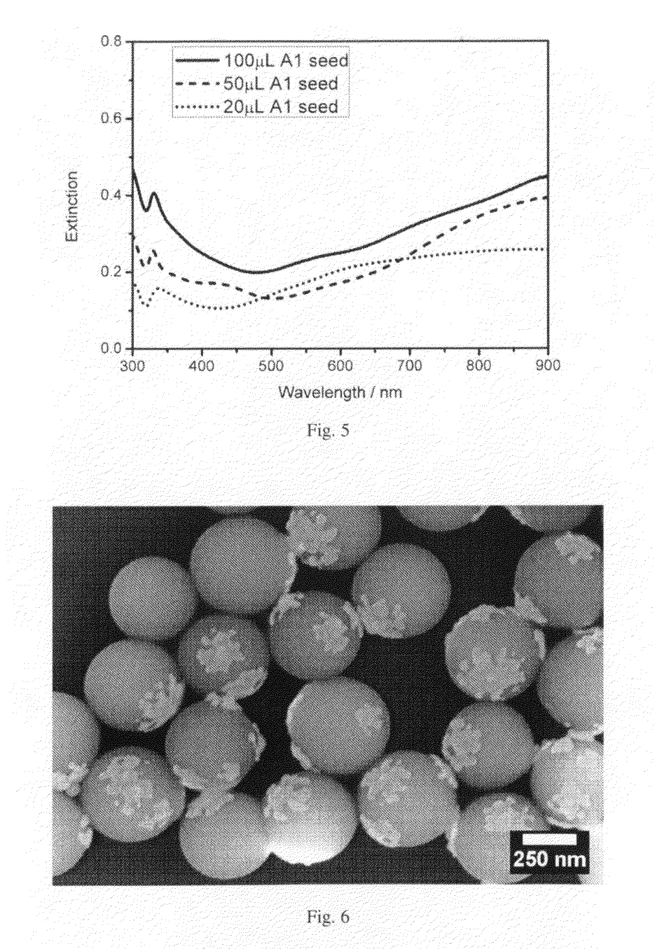

FIGS. 1 to 5 show SEM images of untreated silica, water treated—(seed one day old) of partial silver coatings resulting from additions of 100 μL (FIGS. 1 and 2) or 20 μL (FIGS. 3 and 4) of the seed and extinction spectra (FIG. 5).

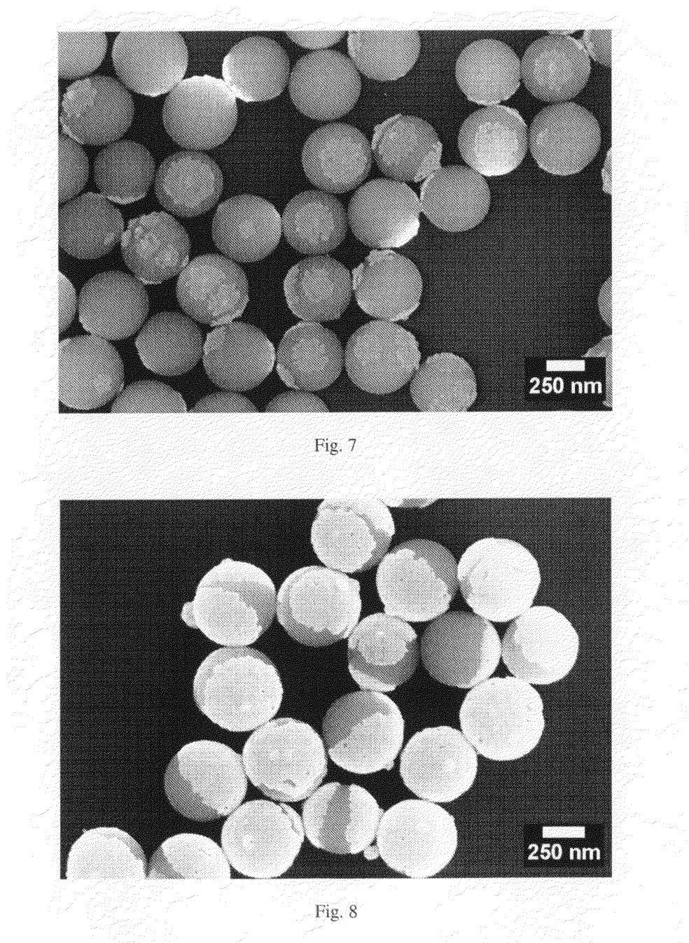

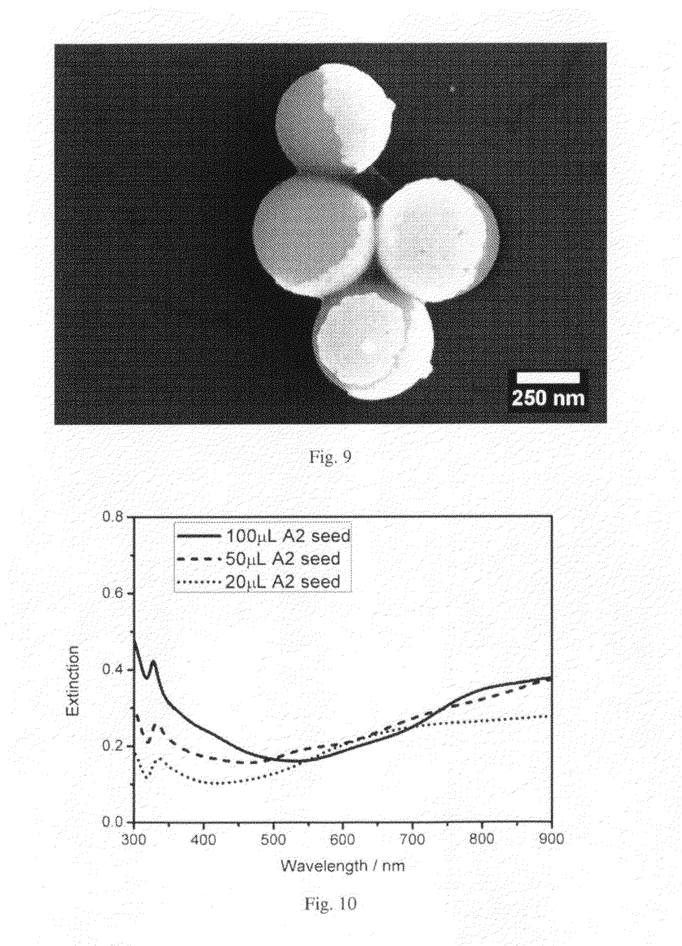

FIGS. 6 to 10 show SEM images of calcined silica, water treated—(seed one day old) of partial silver coatings resulting from additions of 100 μL (FIGS. 6 and 7) or 20 μL (FIGS. 8 and 9) of the seed and extinction spectra (FIG. 10).

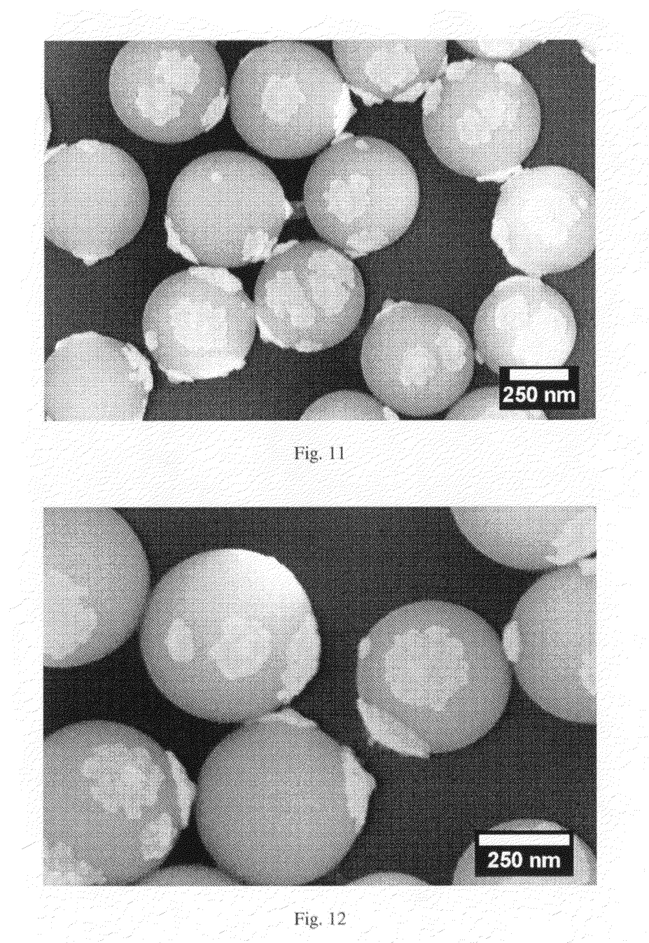

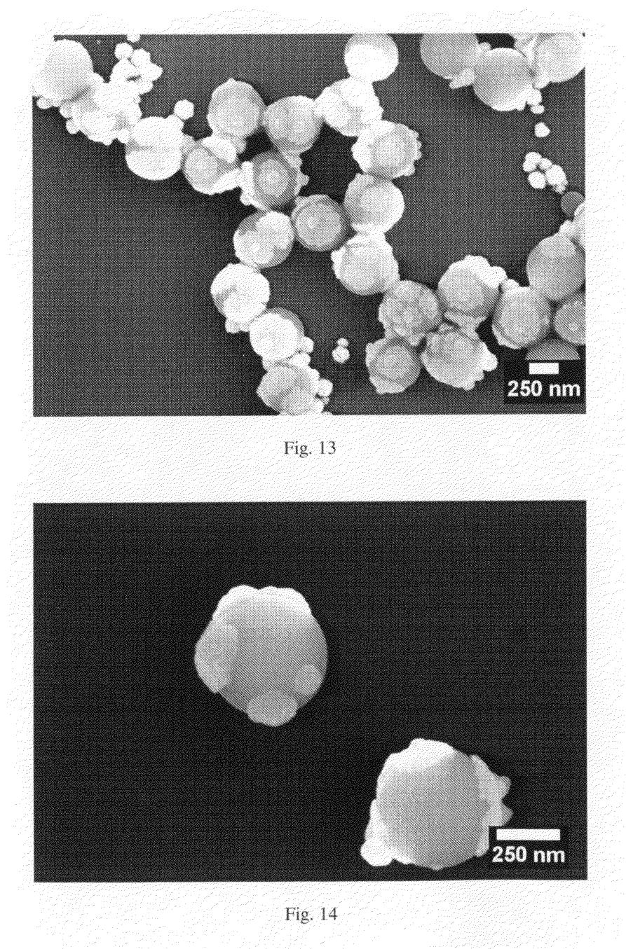

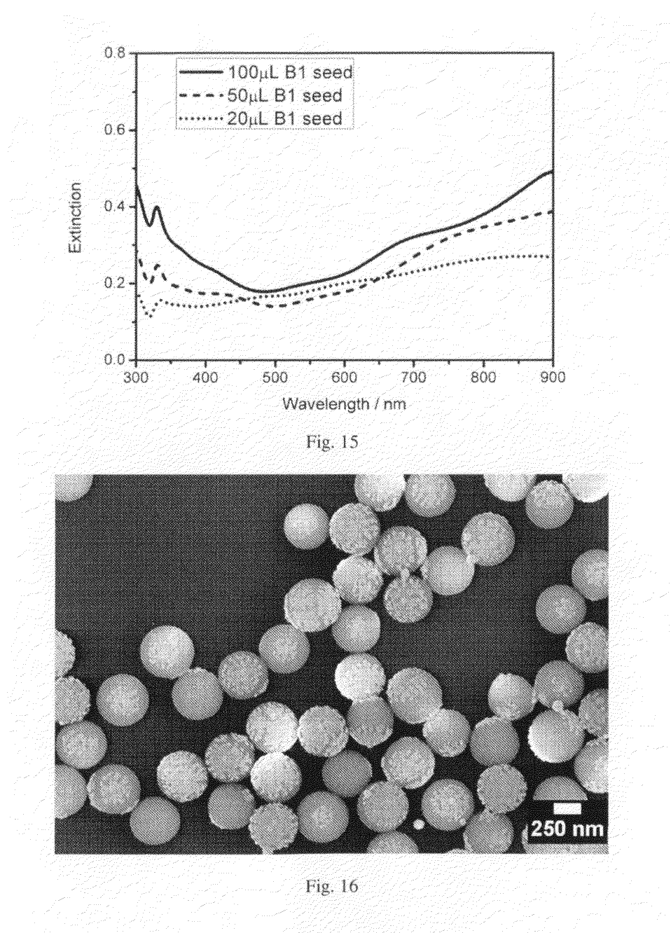

FIGS. 11 to 15 show SEM images of untreated silica, 1,4-Dioxane treated—(seed one day old) of partial silver coatings resulting from additions of 100 μL (FIGS. 11 and 12) or 20 μL (FIGS. 13 and 14) of the seed and extinction spectra (FIG. 15).

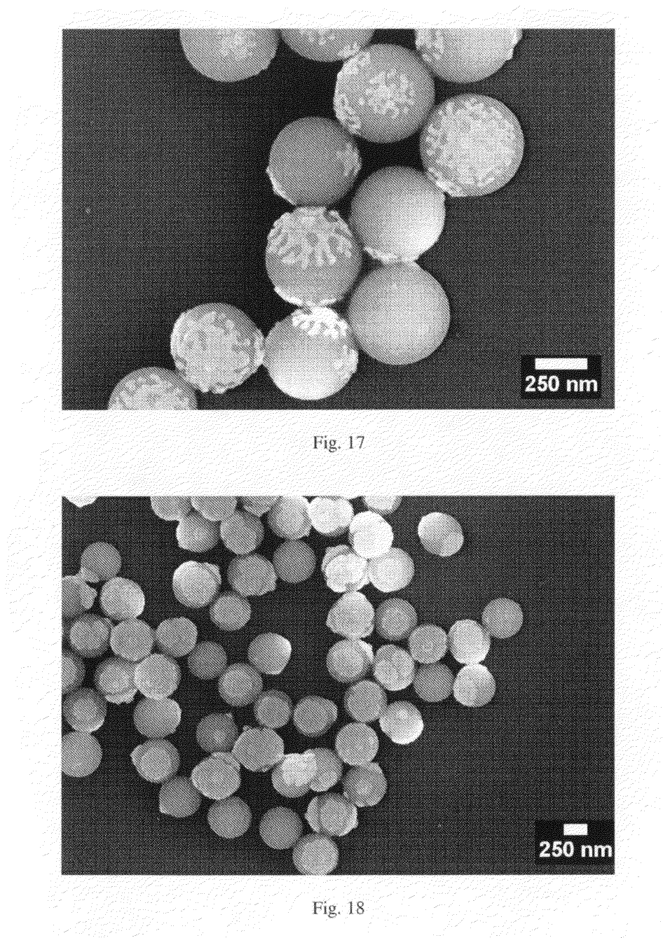

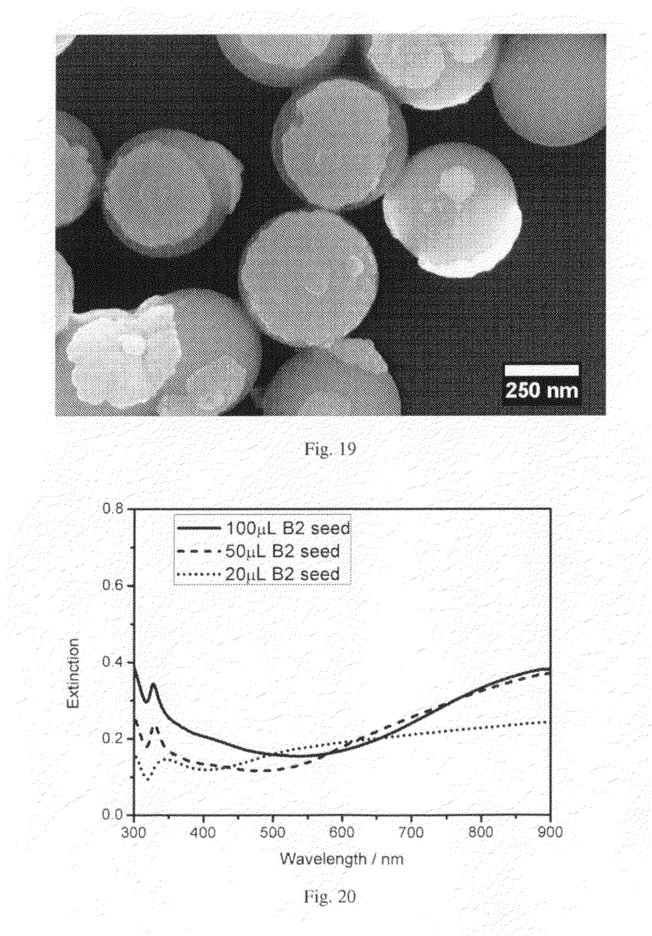

FIGS. 16 to 20 show SEM images of calcined silica, 1,4-Dioxane treated—(seed one day old) of partial silver coatings resulting from additions of 100 μL (FIGS. 16 and 17) or 20 μL (FIGS. 18 and 19) of the seed and extinction spectra (FIG. 20).

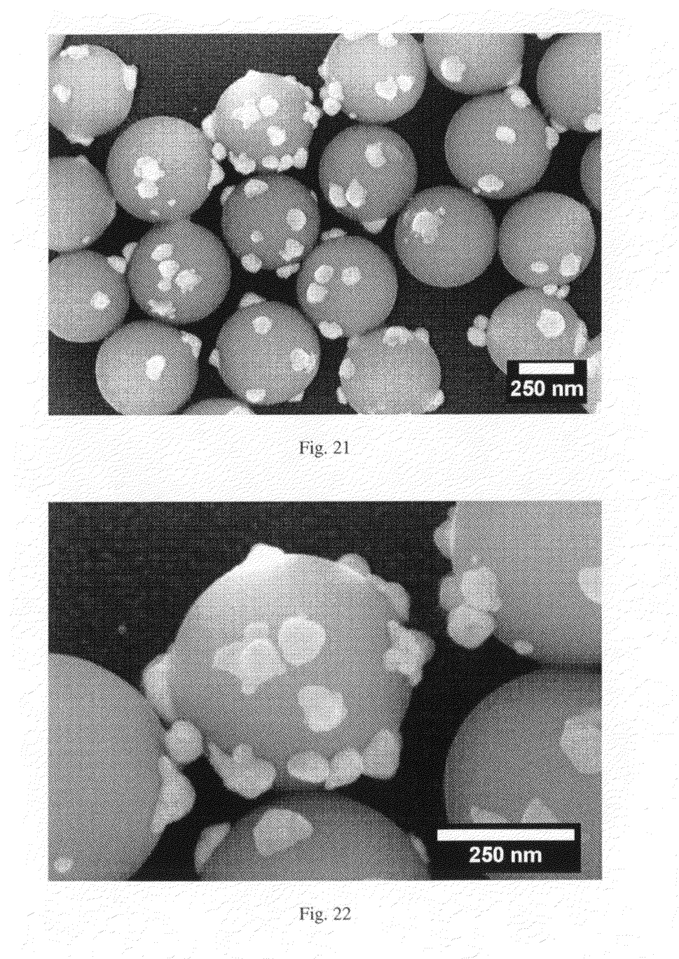

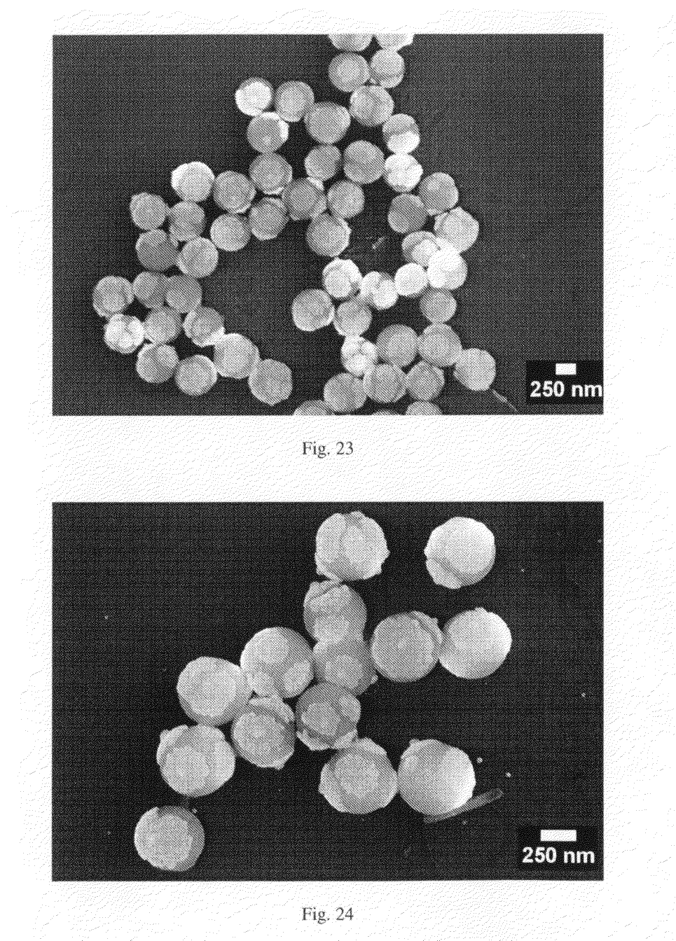

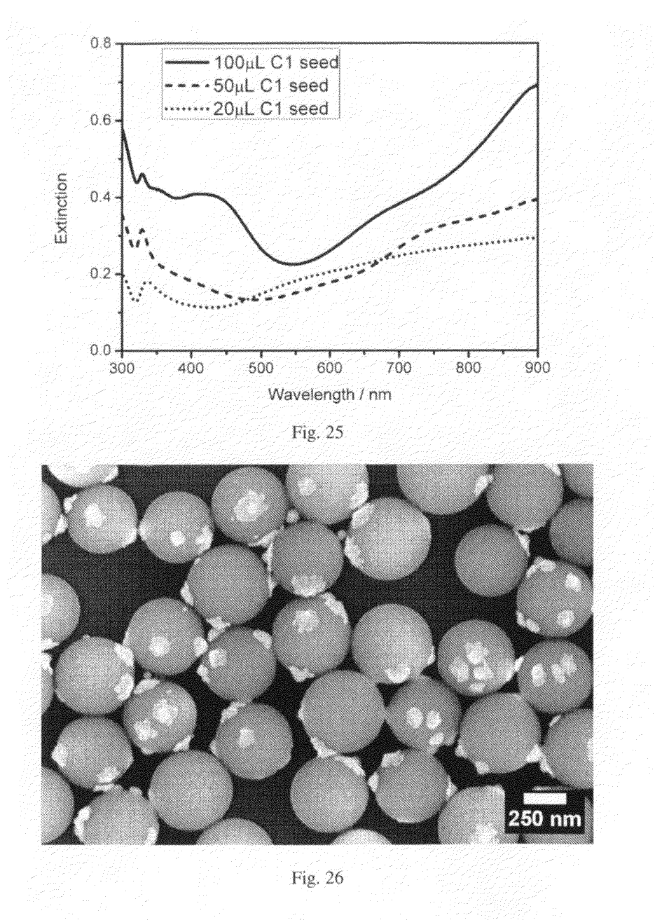

FIGS. 21 to 25 show SEM images of untreated silica, tetrahydrofuran treated—(seed one day old) of partial silver coatings resulting from additions of 100 μL (FIGS. 21 and 22) or 20 μL (FIGS. 23 and 24) of the seed and extinction spectra (FIG. 25).

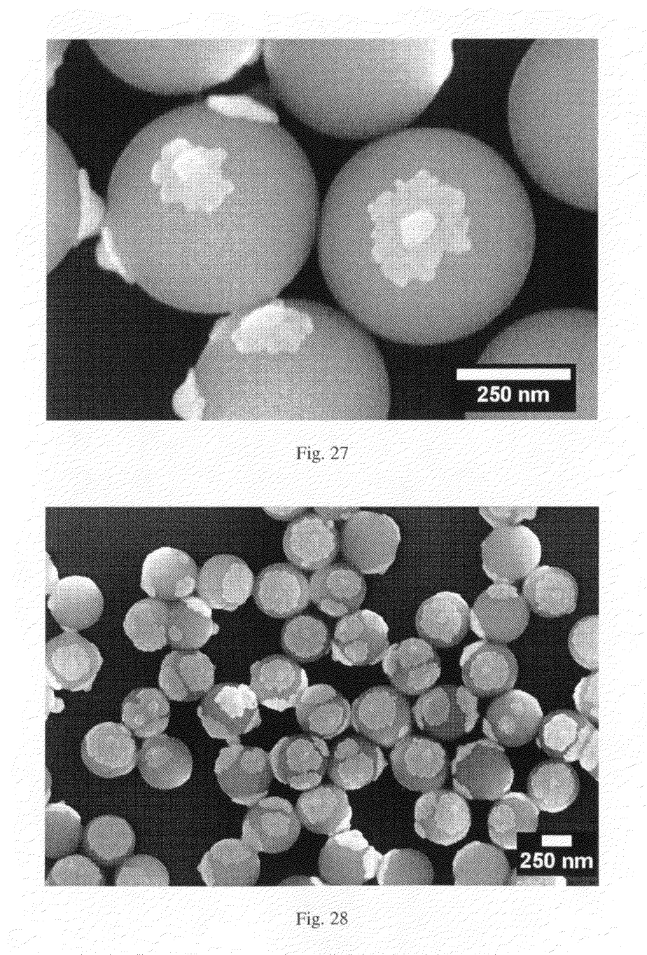

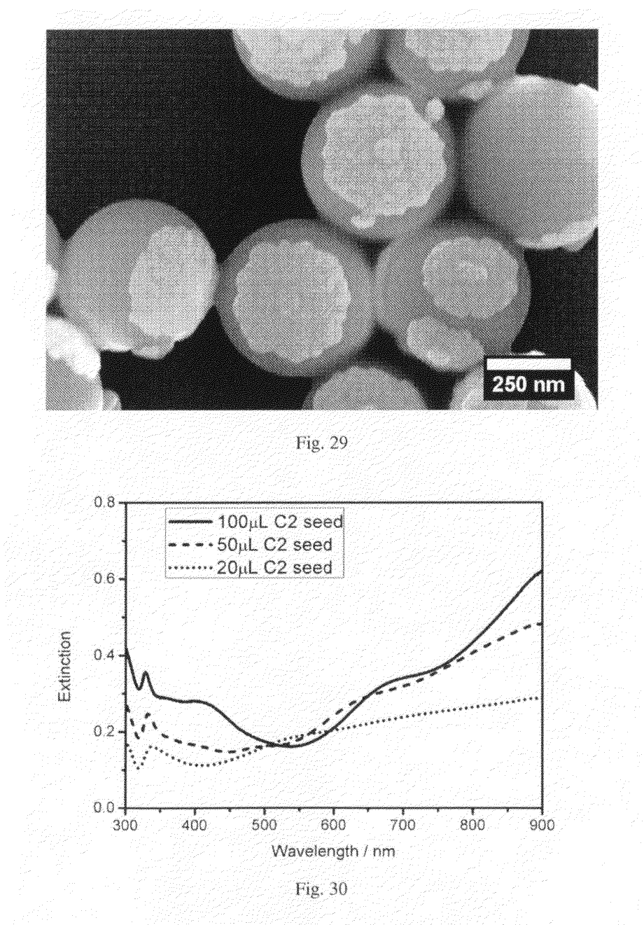

FIGS. 26 to 30 show SEM images of calcined silica, tetrahydrofuran treated—(seed one day old) of partial silver coatings resulting from additions of 100 μL (FIGS. 26 and 27) or 20 μL (FIGS. 28 and 29) of the seed and extinction spectra (FIG. 30).

### EXAMPLE 2

### Asymmetrical Silver Coatings on Silica Particles Coated with Amphiphilic Macromolecules

Materials and Reagents

Monosphere 500 (SiO₂ particles) was purchased from Merck chemical co. Silver nitrate (AgNO₃, >99.9%) and poly (ethylene glycol) were obtained from Sigma chemical co., sodium hydroxide (NaOH), formaldehyde (HCHO, 37%) and triethylamine (C6H15N, >99.5%) from ROTH chemical co, poly (styrene-alt-maleic anhydride) (PSMA) from ACROS chemical co., other reagents from Merck chemical co. All the chemicals were used as received without further purification. The amphiphilic macromolecule (PEG-SA) was an alternating sequence of polyethylene glycol (MW=600) and C₈ alkyl chains and was synthesised in the laboratories of the inventors.

Adsorption of PSMA at Silica Particle Surface

1 g Monosphere 500 powder and 2 g PSMA were mixed in 20 mL 1,4-Dioxane for 6 hours with intense stirring at room temperature. Then the suspension was centrifuged and particles were washed with 1,4-Dioxane. This procedure was repeated for 3 times, finally the particles were dispersed in 20 mL 1,4-Dioxane.

Attachment of PEG-SA to the Silica Sphere Modified by PSMA

2 mL of 50 mg/mL silica suspension modified by PSMA mixed with 0.06 g PEG-SA (S6) in 2 mL 1,4-Dioxane in the presence of catalytic amounts (20 μL) of triethylamine at 60° C. for 4 hours, then suspension was centrifuged and particles were washed with 1,4-Dioxane. This procedure was repeated for 3 times; afterwards particles were washed with water for 3 times and finally dispersed in 2 mL water.

Adsorption of Silver Clusters

0.2 g NaOH and 0.12 g AgNO₃ was dissolved in 1 mL water respectively and then mixed together, the black precipitate of Ag₂O was produced and then washed with water until the pH value of supernatant was around 7. 300 μL 32% ammonium hydroxide (NH₄OH) was added to dissolve Ag₂O, then the ammoniacal silver complex ([Ag (NH₃)₂]OH) was formed. A portion of 120 μL silver complex was added to 2 mL silica-PSMA-PEG-SA mentioned above with addition of 1 mL water; the mixture was stirred for 2 days at room temperature. Then the particles were centrifuged to remove unattached silver nanoparticles and washed with water at least for 3 times. The final volume of modified silica suspension was 2 mL.

Silver Coating

100 μl, 80 μl, 40 μl, 20 μl, 10 μl and 5 μL of 10 times diluted modified silica suspension were added into 5 mL silver nitrate solution (100 μM) respectively, 100 μL formaldehyde (37%) added followed <10 s later by addition of 100 μL 8% ammonium hydroxide with intense stirring. The same trials were repeated except that 100 μL ammonia solution was added before formaldehyde (100 μL)

The same volume (5 mL) of silver nitrate of different concentration (200 μM, 150 μM, 100 μM, 50 μM, 25 μM and 15 μM) mixed with 10 μL diluted particles; the same amount of formaldehyde was added followed by addition of 100 μL ammonia solution.

Influence of addition order

| No | AgNO₃ (mL) | particles (μL) | HCHO (μL) | NH3 (μL) |
|---|---|---|---|---|
| 1 | 5 | 100 | 100 | 100 |
| 2 | 5 | 80 | 100 | 100 |
| 3 | 5 | 40 | 100 | 100 |
| 4 | 5 | 20 | 100 | 100 |
| 5 | 5 | 10 | 100 | 100 |
| 6 | 5 | 5 | 100 | 100 |
| No | AgNO₃ (mL) | particles (μL) | NH₃ (μL) | HCHO (μL) |
| 7 | 5 | 100 | 100 | 100 |
| 8 | 5 | 80 | 100 | 100 |
| 9 | 5 | 40 | 100 | 100 |
| 10 | 5 | 20 | 100 | 100 |
| 11 | 5 | 10 | 100 | 100 |
| 12 | 5 | 5 | 100 | 100 |

Influence of silver nitrate concentration

| No | AgNO₃ (mL) | particles (μL) | HCHO (μL) | NH₃ (μL) |
|---|---|---|---|---|
| 13 | 5 (200 μM) | 10 | 100 | 100 |
| 14 | 5 (150 μM) | 10 | 100 | 100 |
| 5 | 5 (100 μM) | 10 | 100 | 100 |
| 15 | 5 (50 μM) | 10 | 100 | 100 |
| 16 | 5 (25 μM) | 10 | 100 | 100 |
| 17 | 5 (15 μM) | 10 | 100 | 100 |

FIG. 31 shows a SEM image of sample 1—the particles show low coverage thin coating.

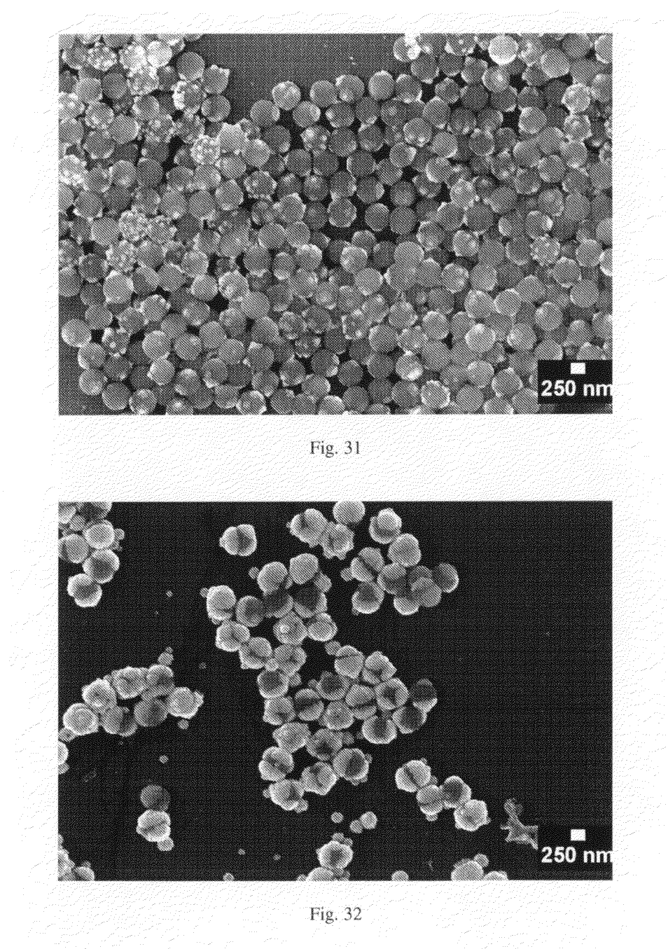

FIG. 32 shows a SEM image of sample 5—the particles show high coverage thin/thick coatings

FIG. 33 shows a SEM image of sample 11—the particles show low coverage thick coatings

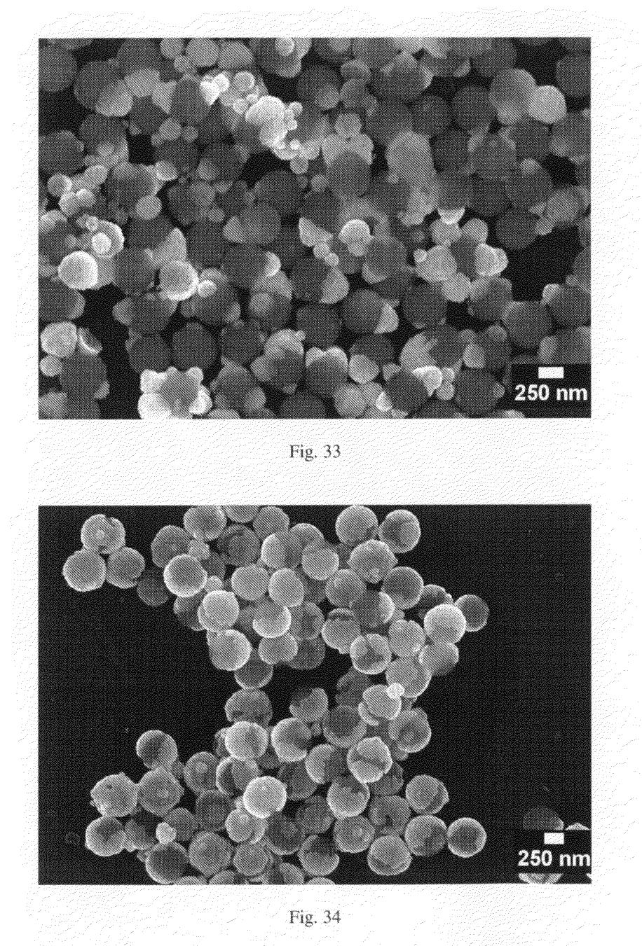

FIG. 34 shows a SEM image of sample 13—the particles show high coverage thin coatings

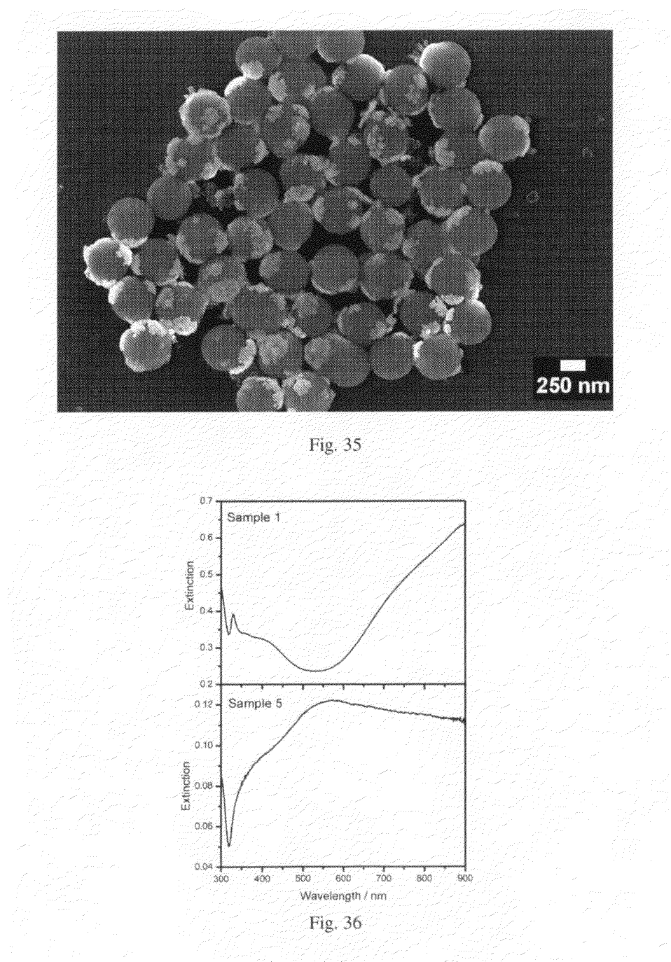

FIG. 35 shows a SEM image of sample 17—the particles show low coverage thin coatings

FIG. 36 shows the optical extinction spectra of sample 1 and sample 5

### EXAMPLE 3

### Asymmetrical Silver Coatings on Silica Particles Coated with Silver Nanoparticles

Adsorption of PSMA at Silica Particle Surface

1 g of Silica powder (Monospher 500, Merck) and 2 g poly (styrene-alt-maleic anhydride) (PSMA) were stirred in 20 mL 1,4-Dioxane for 6 hours. The silica particles were then washed 3 times by centrifugation and 1,4-Dioxane redisperal. The final volume was 20 mL and the concentration of silica was 50 mg/mL. 0.5 mL of this suspension was transferred to high purity water by three centrifugation and water redispersal steps.

Synthesis of Silver Nanoparticles (AgNP)

2 mL silver nitrate (5 mM) was mixed with 133 μL of a 1 mM ethanolic solution of cetyltrimethylammonium bromide (CTAB) under stirring. After 10 minutes fresh sodium borohydride (1%) was added until the colour of the colloidal suspension became yellow green and did not change further. The colloid was washed by centrifugation and high purity water redispersal. The final volume was 500 μL.

Attachment of AgNPs to PSMA-Silica

500 μL silver colloid was added to the 500 μL aqueous PSMA-silica suspension and stirred overnight. The suspension was washed by centrifugation and washing to remove unattached silver nanoparticles. The final volume was 500 μL.

Formation of Asymmetrical Silver Coatings on Silica-PSMA-AgNP Particles

Prior to treatment, a small amount of the silica-PSMA-AgNP suspension (hereafter referred to as the “seed”) was diluted by a factor of 10. Coating experiments comprised adding aliquots of this diluted seed (see table) to 5 mL of a 100 μM silver nitrate solution under vigorous stirring followed by addition of 100 μL 37% aqueous formaldehyde solution and 100 μL 8% aqueous ammonia solution.

| Sample | |
|---|---|
| Code | Seed |
| 080909-1 | 100 μL |
| 080909-2 | 80 μL |
| 080909-3 | 40 μL |
| 080909-4 | 20 μL |
| 080909-5 | 10 μL |

Small aliquots of freshly produced asymmetrical silver-silica suspensions were dried on silicon wafers. These were investigated by SEM. Furthermore, the optical extinction spectra of freshly produced silver-silica suspensions were obtained.

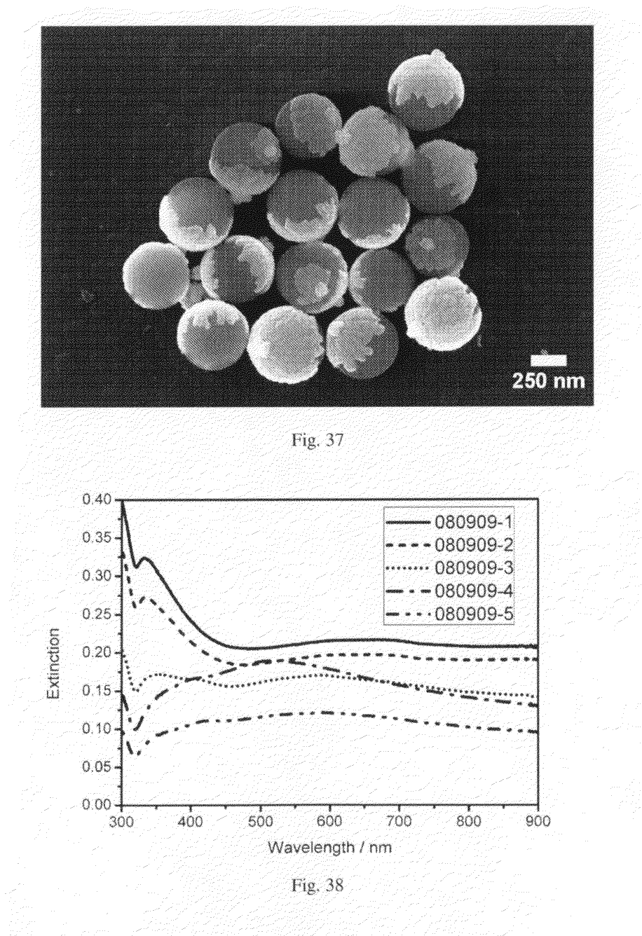

FIG. 37 shows a SEM image of sample 080909-2

FIG. 38 shows extinction spectra of asymmetrically coated silica particles that had been pretreated with PSMA and silver nanoparticles

### EXAMPLE 4

### Effect of Synthetic Procedure on Optical Properties

Silver-impregnated silica spheres were prepared according to Example 1:B1 (500 nm silica spheres, untreated, stored in 1,4 dioxane for 10 days). All preparation steps (including washing) of Example 1 were followed. Silver coatings were produced on the silver-impregnated spheres by mixing certain amounts with silver nitrate, formaldehyde (HCHO) and 8% aqueous ammonia (NH₃) (see table below). The order of addition of formaldehyde and ammonia was varied and in the case of ammonia being added first, the time before formaldehyde was added was varied.

| No. | AgNO₃ | Particles | HCHO | Time | NH₃ |
|---|---|---|---|---|---|
| 081009-31 | 5 mL | 100 μL | 100 μL | <30 s | 100 μL |

| No. | AgNO₃ | Particles | NH₃ | Time | HCHO |
|---|---|---|---|---|---|
| 081009-32 | 5 mL | 100 μL | 100 μL | <30 s | 100 μL |
| 081009-34 | 5 mL | 100 μL | 100 μL | 5 min | 100 μL |
| 081009-36 | 5 mL | 100 μL | 100 μL | 30 min | 100 μL |

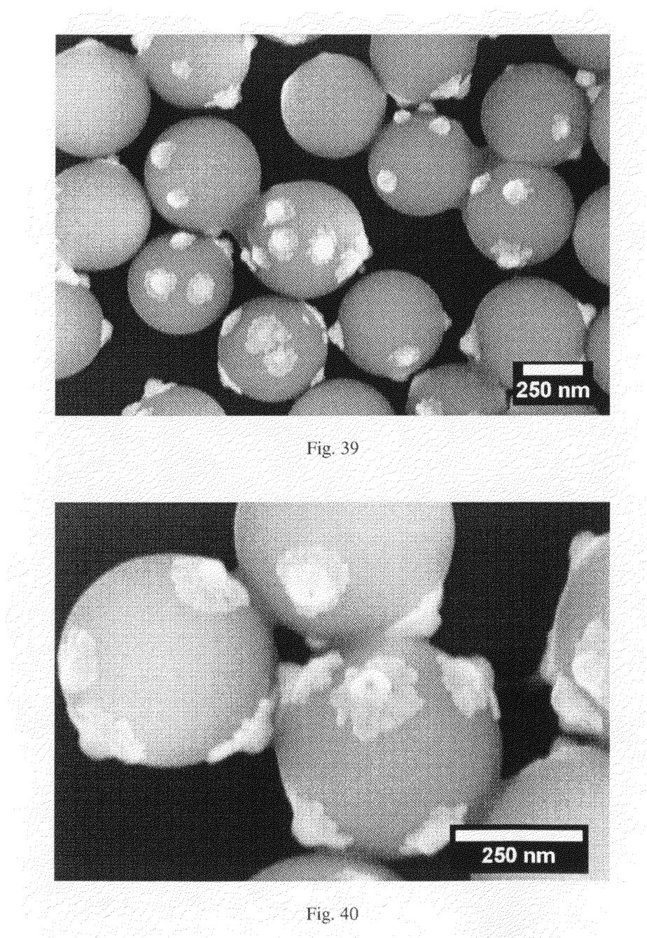

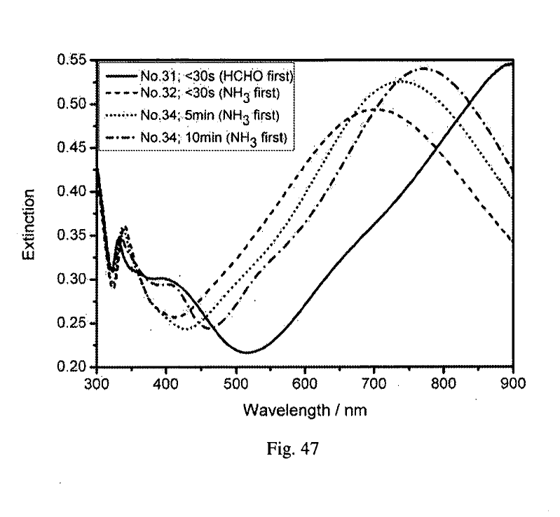

FIGS. 39 to 46 show SEM images for the four samples. It will be noted that when formaldehyde is added first silver caps comprising a hemispheroidal centre and thin, flat edges are formed. On the other hand, when ammonia is added first and formaldehyde a few seconds later, rounded silver caps are formed. If a longer time is left until formaldehyde is added, the caps begin to return to the hemispheroidal centre and thin, flat edge morphology. FIG. 47 shows that these morphological differences have a particular influence on the optical extinction properties of the particles. Most notably, for silver caps with wider thin edges (sample 081009-31), there is an extinction peak in the near infrared. On the other hand, when the caps are rounded (sample 081009-32), the extinction peak is 200 nm blue-shifted in comparison.

FIGS. 39 and 40 show SEM images of sample 081009-31

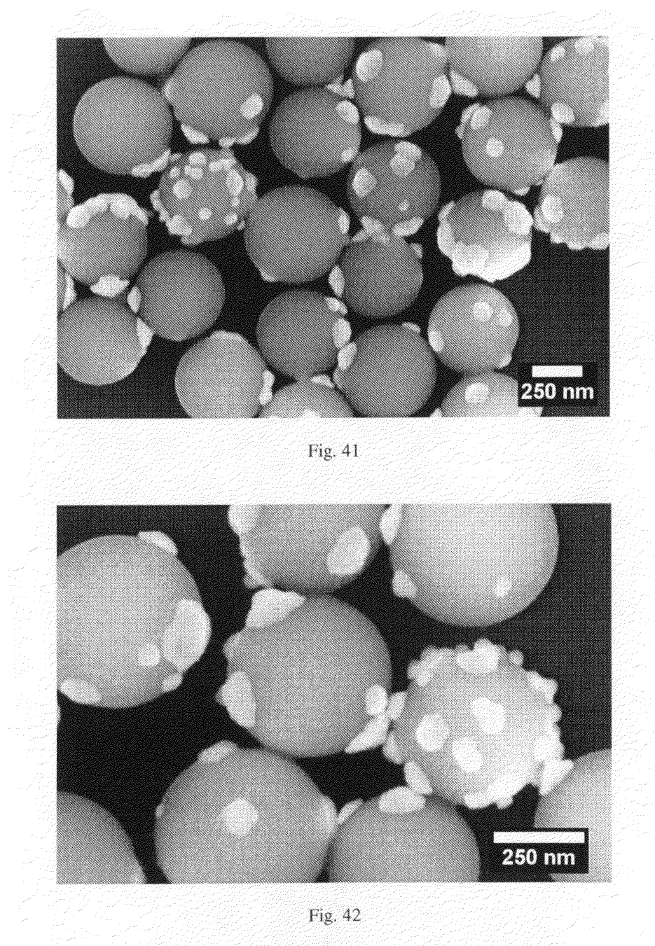

FIGS. 41 and 42 show SEM images of sample 081009-32

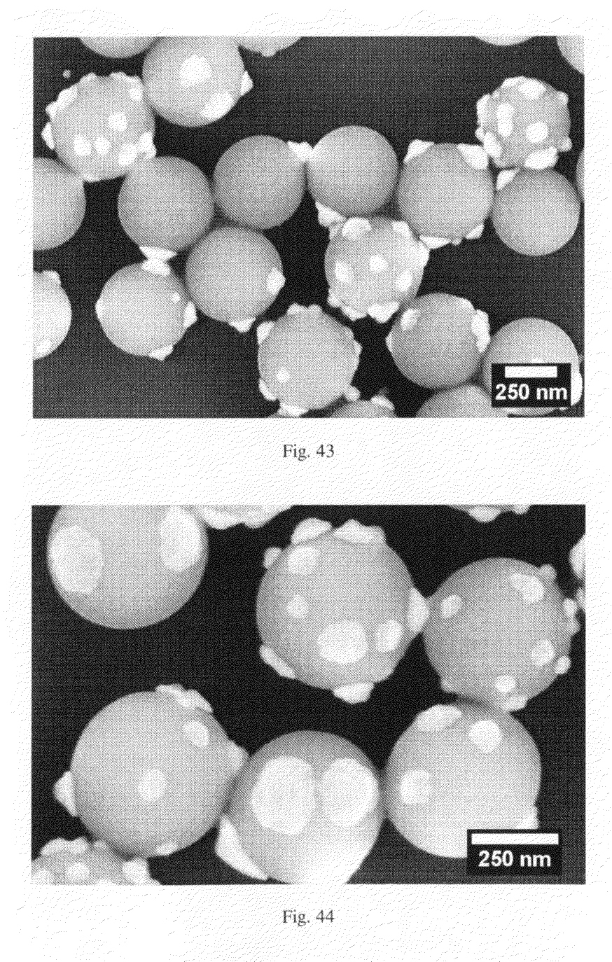

FIGS. 43 and 44 show SEM images of sample 081009-33

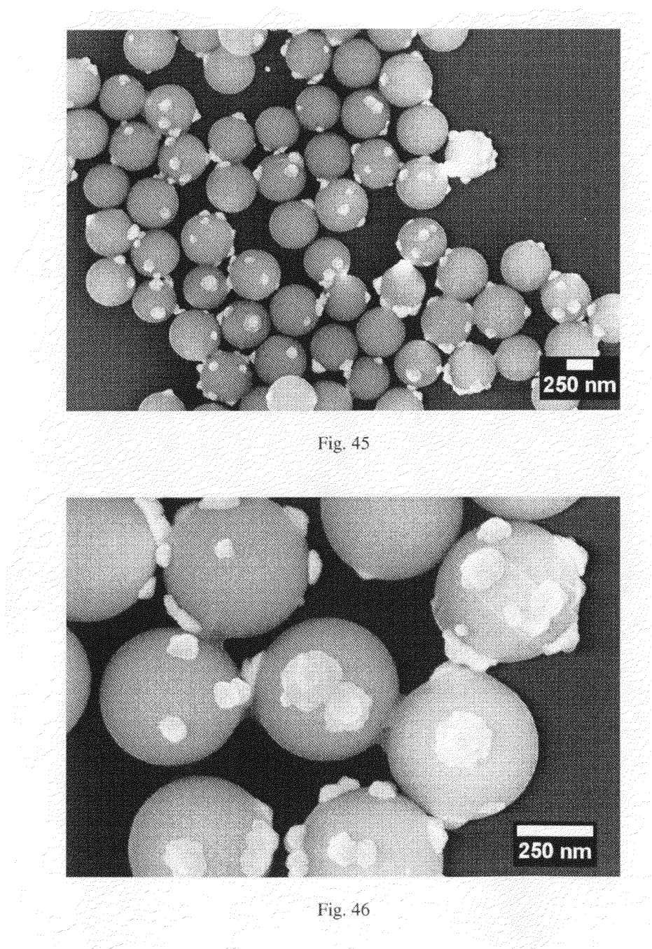

FIGS. 45 and 46 show SEM images of sample 081009-34

FIG. 47 shows UV/VIS extinction curves of samples 081009-31 to 34

### EXAMPLE 5

### Effect of Illumination on Optical Properties

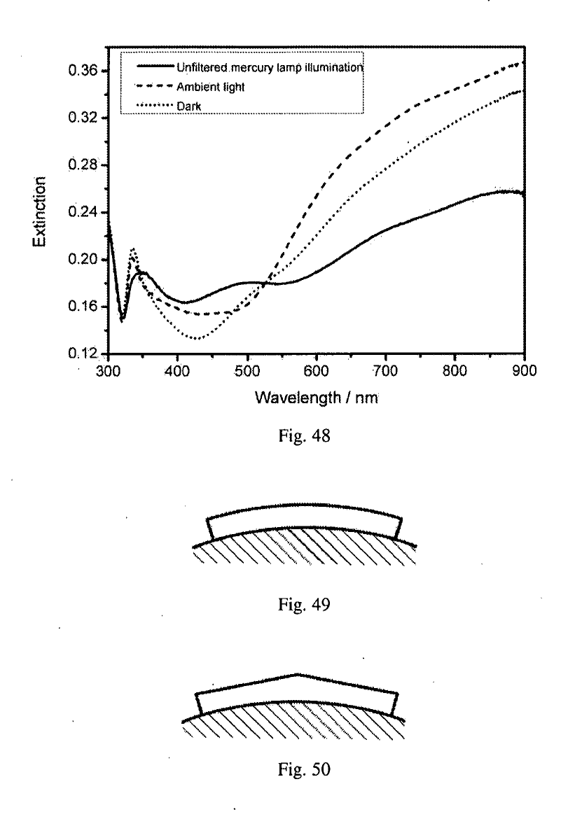

Silver-impregnated silica spheres were prepared according to Example 1:B1 (500 nm silica spheres, untreated, stored in 1,4 dioxane for 10 days) with conditions of illumination being varied during the impregnation step. One sample was stored in the dark for 18 hours, another sample was kept under ambient lighting (sunlight, fluoresecent lamps) for 5 hours and in the dark for 13 hours. A final sample was illuminated with the unfiltered light of a mercury lamp for 2 hours and stored in the dark for 16 hours. All samples were washed as described in the previous examples. Silver coatings were formed according to the same procedure as sample 081009-31 of Example 4. FIG. 48 shows that illumination conditions during the silver-treatment step have a clear influence on the optical properties of the final coated particles.

FIG. 48 shows UV/VIS extinction curves showing the effect of illumination conditions during ethanolamine-silver complex treatment on the optical properties of silver-coated silica particles.

### EXAMPLE 6

Amorphous silica particles were synthesized according to the well-known Stöber process. 5.6 g of tetraethylorthosilicate (VWRInternational GmbH, Germany) was added rapidly to a vigorously stirred mixture of 74 mL of absolute ethanol (VWR International GmbH, Germany), 10 mL of ultrapure water, and 3.2 mL of ammonium hydroxide (32%, MerckGmbH). Stirring was ceased after 10 min, and the reaction was allowed to proceed for 3 h. Following this, the suspension was washed three times by centrifugation and redispersion in absolute ethanol. The silica particles were then dried under vacuum at 60° C. for at least 12 h. Portions of the resulting powder were calcined in air at 800° C. and 1000° C. for 6 h.

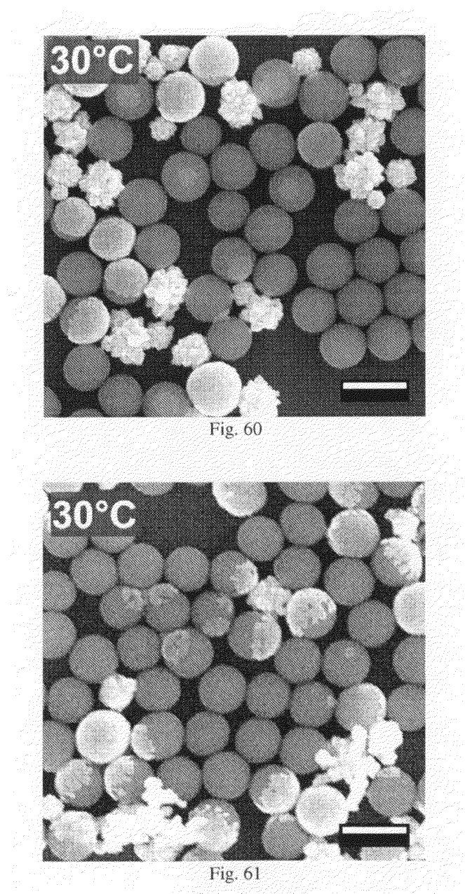

Silica particles were dispersed into Millipore water at a concentration of 50 mg/mL one day before the coating step. A 10 μL portion of this silica suspension was added into 10 mL aqueous silver nitrate solution (100 μM) which was then heated to a temperature of between 30 and 80° C. Following a certain period of aging, 100 μL formaldehyde solution (37% aqueous solution, Carl Roth GmbH, Germany) was added into the suspension under vigorous stirring. This was followed by addition of 50 μL 8% aqueous ammonium hydroxide. The ammonium hydroxide was added dropwise over a period of 10 seconds (unless otherwise stated). Further details of each sample for FIGS. 60-66 are listed below.

| | | Volume of | | Ageing | |
|---|---|---|---|---|---|
| | | silica | Temperature | time in | Addition |
| | Silica calcination | suspension | of 100 μM | 100 μM | time of |
| | temperature | used | AgNO₃ | AgNO₃ | NH₄OH |
| FIG. | [° C.] | [μL] | [° C.] | [min] | [s] |
| 60 | 800° C. | 10 | 30° C. | 0 | 10 |
| 61 | 800° C. | 10 | 30° C. | 30 | 10 |
| 62 | 800° C. | 10 | 50° C. | 0 | 10 |
| 63 | 800° C. | 10 | 50° C. | 30 | 10 |
| 65 | 1000° C. | 10 | 50° C. | 30 | 10 |
| 66 | 1000° C. | 10 | 50° C. | 30 | 25 |

FIG. 60 and FIG. 61 show SEM images (Scalebar=500 nm) of silver patches on silica particles formed by carrying out the silver coating reaction on 800° C. calcined silica at a reaction temperature of 30° C. without (FIG. 60) and with (FIG. 61) 30 minute pre-ageing in silver nitrate at the reaction temperature. It can be seen that the patch yields are similar.

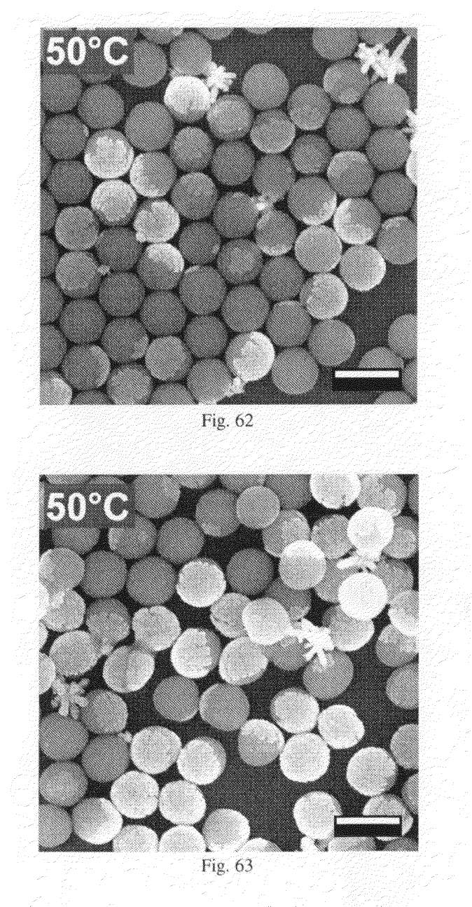

FIG. 62 and FIG. 63 show SEM images (Scalebar=500 nm) of silver patches on silica particles formed by carrying out the silver coating reaction on 800° C. calcined silica at a reaction temperature of 50° C. without (FIG. 62) and with (FIG. 63) 30 minute pre-ageing in silver nitrate at the reaction temperature. It can be seen that the yield in FIG. 62 is slightly better than the 30° C. reaction temperature case (FIG. 60) and the yield in FIG. 63 is significantly better than the 30° C. reaction temperature case (FIG. 61). This shows that both a higher reaction temperature and pre-ageing are preferable.

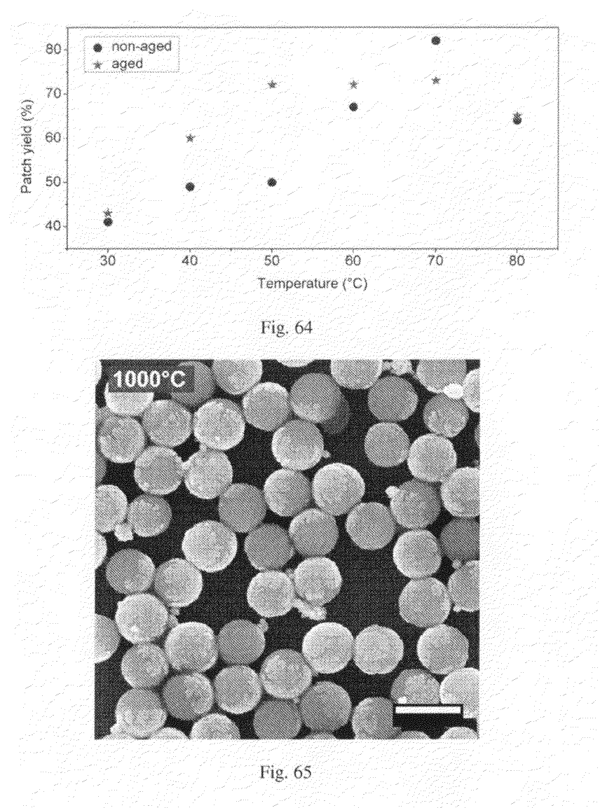

Full data for the effect of reaction temperature and pre-ageing are shown in the table below and on the plot in FIG. 64. It can be seen that as the temperature increases so does the patch yield. It can also be seen that the temperature range 50 to 70° C. is preferred in order to obtain a high patch yield.

| | Non- | |
|---|---|---|
| Reaction | aged | Aged |
| temperature | sample | sample |
| [° C.] | [%] | [%] |
| 30 | 41 | 44 |
| 40 | 50 | 60 |
| 50 | 50 | 72 |
| 60 | 67 | 73 |
| 70 | 82 | 73 |
| 80 | 64 | 65 |

FIG. 65 shows a SEM image (Scalebar=500 nm) of silver patches on silica particles formed by carrying out the silver coating reaction on 1000° C. calcined silica at a reaction temperature of 50° C. with 30 minutes of pre-ageing in silver nitrate solution at the reaction temperature. The patch yield obtained was nearly 100%, indicating that these conditions are preferable. The table below summarizes the effect of the calcination temperature and 30 minute pre-aging in silver nitrate on the patch yields.

| | Non- | |
|---|---|---|
| Calcination | aged | Aged |
| temperature | sample | sample |
| [° C.] | [%] | [%] |
| — | 43 | 44 |
| 800° C. | 50 | 72 |
| 1000° C. | 79 | 99 |

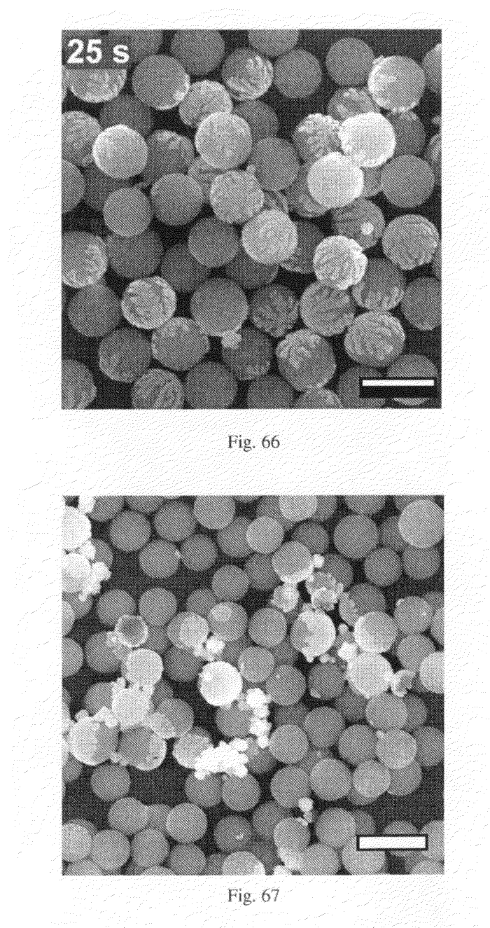

FIG. 66 shows a SEM image (Scalebar=500 nm) of silver patches on silica particles formed by carrying out the silver coating reaction on 1000° C. calcined silica at a reaction temperature of 50° C. with 30 minutes of pre-ageing in silver nitrate solution at the reaction temperature. The ammonia was added here over a period of 25 s (compared to 10 s for the sample in FIG. 65).

It can be seen that this results in a different patch shape i.e. tree-like patches rather than cup-like patches.

### EXAMPLE 7

Amorphous silica particles were synthesized according to the well-known Stöber process. 5.6 g of tetraethylorthosilicate(VWRInternational GmbH, Germany) was added rapidly to a vigorously stirred mixture of 74 mL of absolute ethanol (VWR International GmbH, Germany), 10 mL of ultrapure water, and 3.2 mL of ammonium hydroxide (32%, MerckGmbH). Stirring was ceased after 10 min, and the reaction was allowed to proceed for 3 h. Following this, the suspension was washed three times by centrifugation and redispersion in absolute ethanol. The silica particles were then dried under vacuum at 60° C. for at least 12 h.

Silica particles were dispersed into Millipore water at a concentration of 50 mg/mL and were washed three times in water by centrifugation and redispersion. 0.5 mL portions of this solution were mixed with either monoethanolamine (30 μL) or ammonia (32%, 30 μL) and were stirred for one hour. Following this the dispersions were washed three times in water by centrifugation and redispersion. In a typical growth process, 10 μL portion of silica suspension was added into 10 mL aqueous silver nitrate solution (100 μM) which was then heated to a temperature of 50° C. After 30 minutes aging at this temperature, 100 μL formaldehyde solution (37% aqueous solution, Carl Roth GmbH, Germany) was added into the suspension under vigorous stirring. This was followed by addition of 50 μL 8% ammonium hydroxide. The ammonium hydroxide was added dropwise over a period of 10 seconds.

FIG. 67 shows an SEM image (scalebar=500 nm) of silver patches formed on silica spheres without the silica being pretreated with monoethanolamine or ammonia.

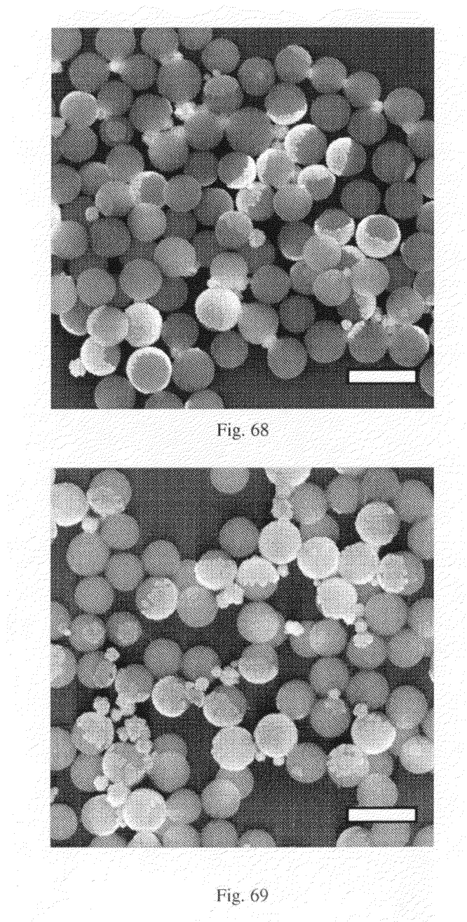

FIG. 68 shows an SEM image (scalebar=500 nm) of silver patches formed on silica spheres with the silica being pretreated with ammonia.

FIG. 69 shows an SEM image (scalebar=500 nm) of silver patches formed on silica spheres with the silica being pretreated with monoethanolamine.

It can be seen that the patch yields (fraction of particles possessing at least on silver patch) are higher and the patches are more uniform in FIGS. 68 and 69 compared to FIG. 67, indicating the benefit obtained by pretreating with ammonia and monoethanolamine.

### EXAMPLE 8

### Removal of Core Silica Particle

Silver patches were produced on silica spheres according to the method used in Example 6 (FIGS. 65 and 66). The dispersion was then centrifuged and the supernatant discarded. The solids were redispersed in 0.5 mL water and 1 mL of 1% aqueous HF was added. After 30 minutes the dispersion was washed by centrifugation and redispersion in water three times.

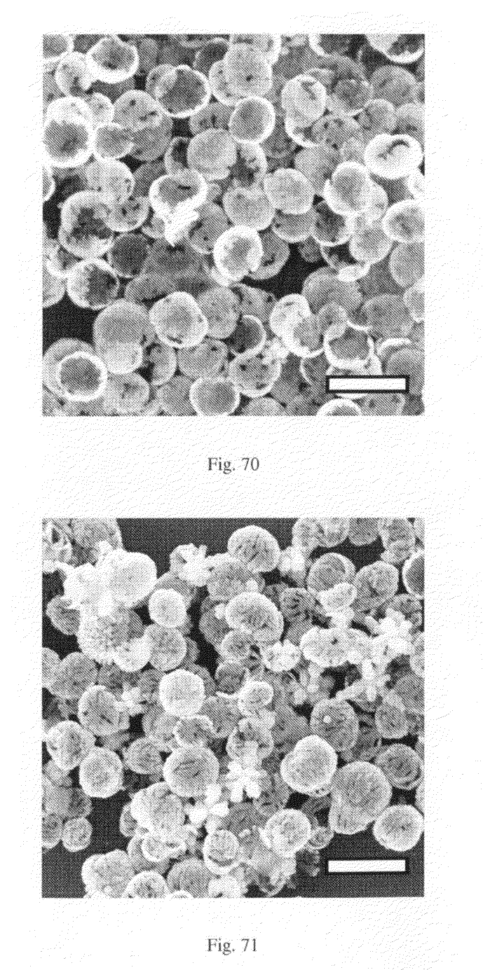

FIG. 70 shows an SEM image (Scalebar=500 nm) of silver cages produced according to the above method for patches synthesized according to the conditions corresponding to FIG. 65.

FIG. 71 shows an SEM image (Scalebar=500 nm) of silver cages produced according to the above method for patches synthesized according to the conditions corresponding to FIG. 66.

It can be seen from FIGS. 70 and 71 that the morphology of the patches (see FIGS. 65 and 66) is preserved even when the silica core particles are dissolved.

1-21. (canceled)

22. A method for synthesis of non-metallic substrates asymmetrically coated with one or more physically separated metallic islands, comprising
(a) Providing a metal oxide substrate,
(b) Treating the substrate with a polar solvent for at least 10 minutes, wherein the polar solvent comprises one or more compounds selected from the group consisting of silver metal ions, silver metal ions and complexing agents and silver metal complexes, and
(c) Treating the substrate subsequent to step (b) with one or more reducing agents selected from the group consisting of formaldehyde, glyoxal, formic acid, glyceraldehyde, glycolaldehyde dimer and glyoxal trimeric dehydrate, and further with one or more additives which are bases and
wherein the proportion of the metal ion or metal complex is in the range from 1×10⁻⁴ to 5×10⁻³% by weight, based on the solution provided in process step (b) and (c), and wherein in steps (b) and (c) the substrate is treated in the respective polar solvent at a temperature from 35 to 95° C.

23. A method for synthesis of non-metallic substrates asymmetrically coated with one or more physically separated metallic islands, comprising
(a) Providing a metal oxide substrate,
(a′) Treating the substrate with a polar solvent that optionally comprises at least one metal complexing agent for at least one minute,
(b) Treating the substrate with a polar solvent for at least 1 minute, wherein the polar solvent comprises one or more compounds selected from the group consisting of silver metal ions, silver metal ions and complexing agents and silver metal complexes and silver metal nanoparticles, and
(c) Treating the substrate subsequent to step (b) with one or more reducing agents selected from the group consisting of formaldehyde, glyoxal, formic acid, glyceraldehyde, glycolaldehyde dimer and glyoxal trimeric dehydrate and further with one or more additives which are bases,
wherein the proportion of the metal is in the range from 1×10⁻⁴ to 5×10⁻³% by weight, based on the solution provided in process step (b) and (c), and
wherein in steps (b) and (c) the substrate is treated in the respective polar solvent at a temperature from 35 to 95° C.

24. The method according to claim 22, wherein
said metal oxide substrate is selected from the group consisting of SiO₂, TiO₂, Al₂O₃, ZrO₂, In₂O_{3,} Fe₂O₃ and Fe₃O₄, and
said one or more additives is selected from the group consisting of ammonium hydroxide, methylamine, dimethylamine, trimethylamine, ethylamine, triethylamine, ethanolamine, diethanolamine, triethanolamine, isopropylamine, ethylenediamine, dimethylethylendiamine, tetramethylethylendiamine and potassium carbonate.

25. The method according to claim 23, wherein
said metal oxide substrate is selected from the group consisting of SiO₂, TiO₂, Al₂O₃, ZrO₂, In₂O_{3,} Fe₂O₃ and Fe₃O₄, and
said one or more additives is selected from the group consisting of ammonium hydroxide, methylamine, dimethylamine, trimethylamine, ethylamine, triethylamine, ethanolamine, diethanolamine, triethanolamine, isopropylamine, ethylenediamine, dimethylethylendiamine, tetramethylethylendiamine and potassium carbonate.

26. The method according to claim 22, wherein step (a) comprises providing a substrate and treating the substrate with a polar solvent.

27. The method according to claim 23, wherein step (a) comprises providing a substrate and treating the substrate with a polar solvent.

28. The method according to claim 22, wherein step (c) is carried out in a second polar solvent, wherein the second polar solvent comprises one or more reducing agents selected from the group consisting of formaldehyde, glyoxal, formic acid, glyceraldehyde, glycolaldehyde dimer and glyoxal trimeric dehydrate, one or more additives selected from the group consisting of bases such as ammonium hydroxide, methylamine, dimethylamine, trimethylamine, ethylamine, triethylamine, ethanolamine, diethanolamine, triethanolamine, isopropylamine, ethylenediamine, dimethylethylendiamine, tetramethylethylendiamine and potassium carbonate, and one or more compounds selected from the group consisting of metal ions, metal ions and complexing agents and metal complexes wherein the metal is selected from the group of Ag and Au and the proportion of the metal is in the range from 1×10⁻⁴ to 5×10⁻³% by weight, based on the solution provided in process step (c).

29. The method according to claim 23, wherein step (c) is carried out in a second polar solvent, wherein the second polar solvent comprises one or more reducing agents selected from the group consisting of formaldehyde, glyoxal, formic acid, glyceraldehyde, glycolaldehyde dimer and glyoxal trimeric dehydrate, one or more additives selected from the group consisting of bases such as ammonium hydroxide, methylamine, dimethylamine, trimethylamine, ethylamine, triethylamine, ethanolamine, diethanolamine, triethanolamine, isopropylamine, ethylenediamine, dimethylethylendiamine, tetramethylethylendiamine and potassium carbonate, and one or more compounds selected from the group consisting of metal ions, metal ions and complexing agents and metal complexes wherein the metal is selected from the group of Ag and Au and the proportion of the metal is in the range from 1×10⁻⁴ to 5×10⁻³% by weight, based on the solution provided in process step (c).

30. The method according to claim 22, wherein the polar solvent is selected from the group consisting of water, tetrahydrofuran, 1,4 dioxane, dimethylsulfoxide, dimethylformamide and C₁ to C₆ alcohols.

31. The method according to claim 23, wherein the polar solvent is selected from the group consisting of water, tetrahydrofuran, 1,4 dioxane, dimethylsulfoxide, dimethylformamide and C₁ to C₆ alcohols.

32. The method according to claim 22, wherein in step (a) at first the substrate is treated by calcination at 500-1100° C.

33. The method according to claim 23, wherein in step (a) at first the substrate is treated by calcination at 500-1100° C.

34. The method according to claim 22 wherein subsequent to step (c) the substrate is removed by chemical or heat treatment.

35. The method according to claim 23, wherein subsequent to step (c) the substrate is removed by chemical or heat treatment.

36. A non-metallic substrate asymmetrically coated with one or more physically separated metallic islands obtainable by the process according to claim 22.

37. A non-metallic substrate asymmetrically coated with one or more physically separated metallic islands obtainable by the process according to claim 23.

38. A method of utilizing the non-metallic substrate according to claim 36 as drug delivery system, in heat management, thermal management, in diagnostics, as a surface-enhanced Raman spectroscopy agent, as pigment, as catalyst, in a light detecting device, in an electronic ink or as chemical sensing device.

39. A method of utilizing the non-metallic substrate according to claim 37 as drug delivery system, in heat management, thermal management, in diagnostics, as a surface-enhanced Raman spectroscopy agent, as pigment, as catalyst, in a light detecting device, in an electronic ink or as chemical sensing device.

40. A method of utilizing the non-metallic substrate according to claim 36 treated at least up to step (b) as a vehicle for the release of metal ions.

41. A method of utilizing the non-metallic substrate according to claim 37 treated at least up to step (b) as a vehicle for the release of metal ions.

42. A method of utilizing a metallic islands obtainable by the process according to claim 34 as drug delivery system, in heat management, thermal management, in diagnostics, as a surface-enhanced Raman spectroscopy agent, as pigment, as catalyst, in a light detecting device, in an electronic ink or as chemical sensing device.

43. A method of utilizing a metallic islands obtainable by the process according to claim 35 as drug delivery system, in heat management, thermal management, in diagnostics, as a surface-enhanced Raman spectroscopy agent, as pigment, as catalyst, in a light detecting device, in an electronic ink or as chemical sensing device.
